SEQUENCE LISTING (62 sequences)

SEQ ID NO: 1 - protein, 198 aa, Clytia gregaria
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRQHLGFWYTLDPNADGLYGNFVP

SEQ ID NO: 2 - protein, 196 aa, Aequorea victoria
MTSKQYSVKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

SEQ ID NO: 3 - protein, 198 aa, Mitrocoma cellularia
MSMGSRYAVKLTTDFDNPKWIARHKHMFNFLDINSNGQINLNEMVHKASNIICKKLGATEEQTKRHQKCVEDFFGGAGLEYDKDTTWPEYIEGWKRLAKTELERHSKNQVTLIRLWGDALFDIIDKDRNGSVSLDEWIQYTHCAGIQQSRGQCEATFAHCDLDGDGKLDVDEMTRQHLGFWYSVDPTCEGLYGGAVPY

SEQ ID NO: 4 - protein, 195 aa, Obelia longissima
MSSKYAVKLKTDFDNPRWIKRHKHMFDFLDINGNGKITLDEIVSKASDDICAKLEATPEQTKRHQVCVEAFFRGCGMEYGKEIAFPQFLDGWKQLATSELKKWARNEPTLIREWGDAVFDIFDKDGSGTITLDEWKAYGKISGISPSQEDCEATFRHCDLDNSGDLDVDEMTRQHLGFWYTLDPEADGLYGNGVP

SEQ ID NO: 5 - protein, 221 aa, Clytia gregaria
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGKLDVDEMTRQHLGFWYTLDPNADGLYGNFVP

SEQ ID NO: 6 - protein, 221 aa, Aequorea victoria
MSVLTPLLLRGLTGSARRLPVPRAKIHSLPPEGKLTSDFDNPRWIGRHKHMFNFLDVNHNGKISLDEMVYKASDIVINNLGATPEQAKRHKDAVEAFFGGAGMKYGVETDWPAYIEGWKKLATDELEKYAKNEPTLIRIWGDALFDIVDKDQNGAITLDEWKAYTKAAGIIQSSEDCEETFRVCDIDESGQLDVDEMTRQHLGFWYTMDPACEKLYGGAVP

SEQ ID NO: 7 - protein, 230 aa, Obelia longissima [sequence omitted]

SEQ ID NO: 8 - protein, 35 aa, Artificial Sequence
  Description of Artificial Sequence Synthetic polypeptide
MSVLTPLLLRGLTGSARRLPVPRAKIHSLPPEGKL

SEQ ID NO: 9 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGDLDVDEMTRQHLGFWYTLDPNADGLYGNFVP

SEQ ID NO: 10 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGDLDVDEMTRQHLGFWYTLDPNADGLYGNFVP

SEQ ID NO: 11 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGELDVDEMTRQHLGFWYTLDPNADGLYGNFVP

SEQ ID NO: 12 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGELDVDEMTRQHLGFWYTLDPNADGLYGNFVP

SEQ ID NO: 13 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGHLDVDEMTRQHLGFWYTLDPNADGLYGNFVP

SEQ ID NO: 14 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGHLDVDEMTRQHLGFWYTLDPNADGLYGNFVP

SEQ ID NO: 15 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGGLDVDEMTRQHLGFWYTLDPNADGLYGNFVP

SEQ ID NO: 16 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGGLDVDEMTRQHLGFWYTLDPNADGLYGNFVP

SEQ ID NO: 17 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGNLDVDEMTRQHLGFWYTLDPNADGLYGNFVP

SEQ ID NO: 18 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGNLDVDEMTRQHLGFWYTLDPNADGLYGNFVP

SEQ ID NO: 19 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGQLDVDEMTRQHLGFWYTLDPNADGLYGNFVP

SEQ ID NO: 20 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGQLDVDEMTRQHLGFWYTLDPNADGLYGNFVP

SEQ ID NO: 21 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGSLDVDEMTRQHLGFWYTLDPNADGLYGNFVP

SEQ ID NO: 22 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGSLDVDEMTRQHLGFWYTLDPNADGLYGNFVP

SEQ ID NO: 23 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGTLDVDEMTRQHLGFWYTLDPNADGLYGNFVP

SEQ ID NO: 24 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGTLDVDEMTRQHLGFWYTLDPNADGLYGNFVP

SEQ ID NO: 25 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGVLDVDEMTRQHLGFWYTLDPNADGLYGNFVP

SEQ ID NO: 26 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGVLDVDEMTRQHLGFWYTLDPNADGLYGNFVP

SEQ ID NO: 27 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGYLDVDEMTRQHLGFWYTLDPNADGLYGNFVP

SEQ ID NO: 28 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGYLDVDEMTRQHLGFWYTLDPNADGLYGNFVP

SEQ ID NO: 29 - protein, 198 aa, Artificial Sequence
  Description of Artificial Sequence Synthetic polypeptide
MADTASKYAVKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGRLDVDEMTRQHLGFWYTLDPNADGLYGNFVP

SEQ ID NO: 30 - protein, 221 aa, Artificial Sequence
  Description of Artificial Sequence Synthetic polypeptide
MSVLTPLLLRGLTGSARRLPVPRAKIHSLPPEGKLRPNFDNPKWVNRHKFMFNFLDINGDGKITLDEIVSKASDDICAKLGATPEQTKRHQDAVEAFFKKIGMDYGKEVEFPAFVDGWKELANYDLKLWSQNKKSLIRDWGEAVFDIFDKDGSGSISLDEWKAYGRISGICSSDEDAEKTFKHCDLDNSGRLDVDEMTRQHLGFWYTLDPNADGLYGNFVP

SEQ ID NO: 31 - protein, 12 aa, Clytia gregaria
DLDNSGKLDVDE

SEQ ID NO: 32 - DNA, 597 bp, Clytia gregaria
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaatttccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatttgggattctggtacaccttggaccccaacgctgatggtctttacggcaattttgttccttaa

SEQ ID NO: 33 - DNA, 591 bp, Aequorea victoria
atgacaagcaaacaatactcagtcaagcttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatttaggattttggtacaccatggatcctgcttgcgaaaagctctacggtggagctgtcccctaa

SEQ ID NO: 34 - DNA, 597 bp, Artificial Sequence
  Description of Artificial Sequence Synthetic polynucleotide
atggctgacactgcatcaaaatacgccgtcaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaatttccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcgatcttgatgttgatgagatgaccagacaacatttgggattctggtacaccttggaccccaacgctgatggtctttacggcaattttgttccttaa

SEQ ID NO: 35 - DNA, 666 bp, Clytia gregaria
atgtccgtcctgacgccgctgctgctgcggggcttgacaggctcggcccggcggctcccagtgccgcgcgccaagatccattcgttgccgccggaggggaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaatttccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcaaacttgatgttgatgagatgaccagacaacatttgggattctggtacaccttggaccccaacgctgatggtctttacggcaattttgttccttaa

SEQ ID NO: 36 - DNA, 666 bp, Aequorea victoria
atgtccgtcctgacgccgctgctgctgcggggcttgacaggctcggcccggcggctcccagtgccgcgcgccaagatccattcgttgccgccggaggggaagcttacatcagacttcgacaacccaagatggattggacgacacaagcatatgttcaatttccttgatgtcaaccacaatggaaaaatctctcttgacgagatggtctacaaggcatctgatattgtcatcaataaccttggagcaacacctgagcaagccaaacgacacaaagatgctgtagaagccttcttcggaggagctggaatgaaatatggtgtggaaactgattggcctgcatatattgaaggatggaaaaaattggctactgatgaattggagaaatacgccaaaaacgaaccaacgctcatccgtatatggggtgatgctttgtttgatatcgttgacaaagatcaaaatggagccattacactggatgaatggaaagcatacaccaaagctgctggtatcatccaatcatcagaagattgcgaggaaacattcagagtgtgcgatattgatgaaagtggacaactcgatgttgatgagatgacaagacaacatttaggattttggtacaccatggatcctgcttgcgaaaagctctacggtggagctgtcccctaa

SEQ ID NO: 37 - DNA, 666 bp, Artificial Sequence
  Description of Artificial Sequence Synthetic polynucleotide
atgtccgtcctgacgccgctgctgctgcggggcttgacaggctcggcccggcggctcccagtgccgcgcgccaagatccattcgttgccgccggaggggaaactcagacccaacttcgacaacccaaaatgggtcaacagacacaaatttatgttcaactttttggacattaacggcgacggaaaaatcactttggatgaaatcgtctccaaagcttcggatgacatttgcgccaaacttggagcaacaccagaacagaccaaacgtcaccaggatgctgtcgaagctttcttcaaaaagattggtatggattatggtaaagaagtcgaatttccagcttttgttgatggatggaaagaactggccaattatgacttgaaactttggtctcaaaacaagaaatctttgatccgcgactggggagaagctgttttcgacatttttgacaaagacggaagtggctcaatcagtttggacgaatggaaggcttatggacgaatctctggaatctgctcatcagacgaagacgccgaaaagaccttcaaacattgcgatttggacaacagtggcgatcttgatgttgatgagatgaccagacaacatttgggattctggtacaccttggaccccaacgctgatggtctttacggcaattttgttccttaa

SEQ ID NO: 38 - DNA, 43 bp, Artificial Sequence
  Description of Artificial Sequence Synthetic primer
gcgatttggacaacagtggcgatcttgatgttgatgagatgac

SEQ ID NO: 39 - DNA, 43 bp, Artificial Sequence
  Description of Artificial Sequence Synthetic primer
gtcatctcatcaacatcaagatcgccactgttgtccaaatcgc

SEQ ID NO: 40 - DNA, 43 bp, Artificial Sequence
  Description of Artificial Sequence Synthetic primer
gcgatttggacaacagtggcgaacttgatgttgatgagatgac

SEQ ID NO: 41 - DNA, 43 bp, Artificial Sequence
  Description of Artificial Sequence Synthetic primer
gtcatctcatcaacatcaagttcgccactgttgtccaaatcgc

SEQ ID NO: 42 - DNA, 43 bp, Artificial Sequence
  Description of Artificial Sequence Synthetic primer
gcgatttggacaacagtggcggacttgatgttgatgagatgac

SEQ ID NO: 43 - DNA, 43 bp, Artificial Sequence
  Description of Artificial Sequence Synthetic primer
gtcatctcatcaacatcaagtccgccactgttgtccaaatcgc

SEQ ID NO: 44 - DNA, 43 bp, Artificial Sequence
  Description of Artificial Sequence Synthetic primer
gcgatttggacaacagtggcaatcttgatgttgatgagatgac

SEQ ID NO: 45 - DNA, 43 bp, Artificial Sequence
  Description of Artificial Sequence Synthetic primer
gtcatctcatcaacatcaagattgccactgttgtccaaatcgc

SEQ ID NO: 46 - DNA, 43 bp, Artificial Sequence
  Description of Artificial Sequence Synthetic primer
gcgatttggacaacagtggccaacttgatgttgatgagatgac

SEQ ID NO: 47 - DNA, 43 bp, Artificial Sequence; identical to SEQ ID NO: 41
  Description of Artificial Sequence Synthetic primer

SEQ ID NO: 48 - DNA, 43 bp, Artificial Sequence
  Description of Artificial Sequence Synthetic primer
gcgatttggacaacagtggcgtacttgatgttgatgagatgac

SEQ ID NO: 49 - DNA, 43 bp, Artificial Sequence
  Description of Artificial Sequence Synthetic primer
gtcatctcatcaacatcaagtacgccactgttgtccaaatcgc

SEQ ID NO: 50 - DNA, 43 bp, Artificial Sequence
  Description of Artificial Sequence Synthetic primer
gcgatttggacaacagtggcagtcttgatgttgatgagatgac

SEQ ID NO: 51 - DNA, 43 bp, Artificial Sequence
  Description of Artificial Sequence Synthetic primer
gtcatctcatcaacatcaagactgccactgttgtccaaatcgc

SEQ ID NO: 52 - DNA, 43 bp, Artificial Sequence
  Description of Artificial Sequence Synthetic primer
gcgatttggacaacagtggcacacttgatgttgatgagatgac

SEQ ID NO: 53 - DNA, 43 bp, Artificial Sequence
  Description of Artificial Sequence Synthetic primer
gtcatctcatcaacatcaagtgtgccactgttgtccaaatcgc

SEQ ID NO: 54 - DNA, 43 bp, Artificial Sequence
  Description of Artificial Sequence Synthetic primer
gcgatttggacaacagtggctatcttgatgttgatgagatgac

SEQ ID NO: 55 - DNA, 43 bp, Artificial Sequence
  Description of Artificial Sequence Synthetic primer
gtcatctcatcaacatcaagatagccactgttgtccaaatcgc

SEQ ID NO: 56 - DNA, 43 bp, Artificial Sequence
  Description of Artificial Sequence Synthetic primer
gcgatttggacaacagtggcagacttgatgttgatgagatgac

SEQ ID NO: 57 - DNA, 43 bp, Artificial Sequence
  Description of Artificial Sequence Synthetic primer
gtcatctcatcaacatcaagtctgccactgttgtccaaatcgc

SEQ ID NO: 58 - DNA, 43 bp, Artificial Sequence
  Description of Artificial Sequence Synthetic primer
gcgatttggacaacagtggccatcttgatgttgatgagatgac

SEQ ID NO: 59 - DNA, 43 bp, Artificial Sequence
  Description of Artificial Sequence Synthetic primer
gtcatctcatcaacatcaagatggccactgttgtccaaatcgc

SEQ ID NO: 60 - DNA, 29 bp, Artificial Sequence
  Description of Artificial Sequence Synthetic primer
gccgccaccatgagcctccccaattcctc

SEQ ID NO: 61 - DNA, 28 bp, Artificial Sequence
  Description of Artificial Sequence Synthetic primer
tcatcaggagcgaatatgcagaattctc

SEQ ID NO: 62 - protein, 6 aa, Artificial Sequence
  Description of Artificial Sequence Synthetic 6xHis tag
HHHHHH

(19) United States
(12) Patent Application Publication
(10) Pub. No.: US 2011/0081661 A1
(43) Pub. Date: Apr. 7, 2011

(54) Modified photoproteins with increased affinity for calcium and enhanced bioluminescence and uses thereof

(75) Inventors: Lucas Armstrong, Temecula, CA (US); Ming Li, Irvine, CA (US); Matthew Hsu, Escondido, CA (US)

(73) Assignee: Millipore Corporation, Billerica, MA (US)

(21) Appl. No.: 12/803,767

(22) Filed: Jul. 6, 2010

Related U.S. Application Data
(60) Provisional application No. 61/276,875, filed on Sep. 17, 2009.
(60) Provisional application No. 61/270,826, filed on Jul. 14, 2009.

(51) Int. Cl. G01N 33/53; C07K 14/00; C07H 21/00; C12N 15/63; C12N 5/10

(52) U.S. Cl. 435/7.21; 530/350; 536/23.1; 435/320.1; 435/325

(57) ABSTRACT

The present invention provides modified photoproteins, e.g., modified Clytin, having an increased affinity for calcium as well as an enhanced bioluminescence and their use as calcium indicators in reporter gene systems and in cell-based assays.

### RELATED APPLICATIONS

This patent application claims the benefit of priority of provisional patent application No. 61/276,875, filed Sep. 17, 2009 and provisional patent application No. 61/270,826, filed Jul. 14, 2009, the entire contents of each provisional application are incorporated by reference in their entirety.

### FIELD OF THE INVENTION

The present invention provides modified photoproteins, e.g., modified Clytin, having an increased affinity for calcium as well as an enhanced bioluminescence and their use as calcium indicators in reporter gene systems and in cell-based assays.

### BACKGROUND OF THE INVENTION

Several photoproteins that have been reported to emit light upon reacting with Ca²⁺ have been isolated from organisms to date, including Aequorin, Halistaurin, Obelin, Mnemiospin, Clytin and Berovin. In general, all of the aforementioned photoproteins are relatively small in size and are thought to contain a common organic substrate (coelenterazine) and molecular oxygen bound in the form of a complex.

Aequorin has been the most widely studied Ca²⁺ activated photoprotein, isolated from the hydroid Aequorea victoria. In case, of Aequorin, the binding of Ca²⁺ causes a conformational change in the protein, converting the protein to an enzyme which catalyzes the oxidation of coelenterazine by oxygen, with the emission of light (i.e., λmax=470 nm). Aequorin has been used for detecting calcium flux in cells, particularly, as mediated by G protein-coupled receptors (GPCRs), e.g., as described in Stables et al. (Anal. Biochem., 252: 115-126 (1997)). Further, Aequorin-mediated luminescent calcium assays have been employed in high throughput screening for GPCRs, e.g., as described in Ungrin et al. (Anal. Biochem., 272: 34-42 (1999)). Additionally, Aequorin-expressing cells have also been used in drug screening assays, e.g., as described in U.S. Pat. No. 6,872,538.

Although, calcium-activated photoproteins such as, for example, Aequorin, are being used for detecting calcium flux stimulated by GPCRs, for example, and in drug screening, the affinity of Aequorin for calcium is rather low (i.e., only around 7 μM) relative to the cytosolic concentrations of calcium that are induced by receptors (e.g., in the 0.1 μM to 0.2 μM range). Further, although, mitochondrially targeted Aequorin appears to yield a better signal after GPCR simulation, the affinity for calcium is affected by the heterogeneity of mitochondrial calcium accumulation and generally lower amount of Aequorin expression in the mitochondria versus the cytosol.

Similarly, other calcium-activated photoproteins such as, for example, Obelin and Clytin, have also been reported to have lower affinity for calcium and/or low level of light emission. See, e.g., Inouye and Sahara, Protein Express. Purif., 53: 384-389 (2007); Bovolenta et al., J. Biomol. Screen, 12: 694-704 (2007).

### SUMMARY OF THE INVENTION

The present invention provides, at least in part, modified photoproteins, e.g., modified forms of Clytin, which have an increased affinity for intracellular calcium and/or exhibit enhanced bioluminescence relative to the wild-type (wt) photoproteins that are known in the art, e.g., wt-Aequorin and/or wt-Clytin and/or wt-Obelin. The present invention further provides uses of such photoproteins in cell-based assays for detection of calcium flux, e.g., as stimulated by GPCRs and also their use in drug discovery.

In some embodiments according to the invention, a modified photoprotein is provided, which comprises an amino acid sequence comprising at least one amino acid modification in an EF hand III domain of wt-Clytin, the amino acid sequence of which is set forth in SEQ ID NO:1, where the modified photoprotein exhibits an increased affinity for intracellular calcium and increased bioluminescence relative to a photoprotein comprising an amino acid sequence set forth in SEQ ID NO:1.

In some embodiments, a modified photoprotein according to the invention comprises an amino acid sequence comprising at least the lysine at position 168 of the amino acid sequence set forth in SEQ ID NO:1 replaced with an amino acid other than a histidine, an arginine and a lysine; where the modified photoprotein exhibits an increased affinity for intracellular calcium and enhanced bioluminescence relative to the photoprotein comprising the amino acid sequence set forth in SEQ ID NO:1.

In a particular embodiment, a modified photoprotein is provided which comprises an amino acid sequence comprising at least the lysine at position 168 of the amino acid sequence set forth in SEQ ID NO:1 replaced with an aspartic acid; where the modified photoprotein comprises increased affinity for intracellular calcium and enhanced bioluminescence relative to both a photoprotein comprising the amino acid sequence set forth in SEQ ID NO:1 and a photoprotein comprising the amino acid sequence set forth in SEQ ID NO:2.

In various embodiments, the modified photoproteins of the present invention exhibit an increased affinity for intracellular calcium relative to wt-Clytin and/or wt-Aequorin. In some embodiments, a modified photoprotein comprises an EC50 value of 500 nM or lower for intracellular calcium, where the modified photoprotein does not comprise the amino acid sequence set forth in SEQ ID NO:2 or a variant thereof.

Modified photoproteins encompassed by the present invention include a photoprotein comprising an amino acid sequence selected from the group consisting of: SEQ ID NO:9 (K168D); SEQ ID NO:11 (K168E), SEQ ID NO:15 (K168G); SEQ ID NO:17 (K168N); SEQ ID NO:19 (K168Q); SEQ ID NO:21 (K168S); SEQ ID NO:23 (K168T); SEQ ID NO:25 (K168V) and SEQ ID NO:27 (K168Y).

In various embodiments, modified photoproteins encompassed by the present invention exhibit an increased affinity for intracellular calcium relative to wt-Clytin, the amino acid sequence for which is set forth in SEQ ID NO:1. In some embodiments, a modified photoprotein according to the present invention exhibits a 1.5%. or a 2%, or a 3%, or a 4%, or a 5%, or a 10%, or a 20%, or a 25%, or a 30%, or a 40%, or a 50%, or a 60%, or a 70%, or a 80%, or a 90%, or greater than 90%, increase in affinity for intracellular calcium relative to a photoprotein comprising the amino acid sequence set forth in SEQ ID NO:1.

Additionally, in various embodiments, modified photoproteins encompassed by the present invention exhibit an enhanced bioluminescence relative to wt-Clytin. In some embodiments, a modified photoprotein according to the invention exhibits a 1.5%. or a 2%, or a 3%, or a 4%, or a 5%, or a 10%, or a 20%, or a 25%, or a 30%, or a 40%, or a 50%, or a 60%, or a 70%, or a 80%, or a 90%, or greater than 90%, increase in bioluminescence relative to a photoprotein comprising the amino acid sequence set forth in SEQ ID NO:1.

Various photoproteins encompassed by the present invention exhibit both an increased affinity for intracellular calcium as well as enhanced bioluminescence relative to one or both of wt-Clytin and wt-Aequorin.

In some affinity for intracellular calcium as well as bioluminescence exhibited by one or more photoproteins of the present invention is measured in a cell transfected with a nucleic acid molecule encoding the modified photoprotein. Exemplary cells include, but are not limited to, a CHO cell, a HEK293T cell, a HeLa cell, an NIH3T3 cell and a U-2OS cell.

Also encompassed by the present invention are nucleic acid molecules encoding the photoproteins of the invention and vectors comprising such nucleic acid molecules. In further embodiments, a mammalian cells transfected with a nucleic acid encoding a modified photoprotein according to the invention is provided.

In further embodiments, methods of using the modified photoproteins encompassed by the present invention are provided.

In some embodiments, an in vitro method for detecting calcium flux in a cell is provided. Such a method comprises the steps of: a) providing a cell expressing a modified photoprotein as described herein; b) contacting the cell with an agent which causes calcium flux; and c) detecting the photoprotein bioluminescence, where the bioluminescence is indicative of calcium flux.

In further embodiments, a method for screening for compounds which modulate GPCR activity or ion channel is provided, where such a method comprises the steps of: a) providing a cell expressing a modified photoprotein as described herein; b) contacting the cell with a candidate compound; and c) detecting the photoprotein bioluminescence, where a change in the photoprotein bioluminescence in the presence of the candidate compound indicates that the compound modulates GPCR activity or ion channel activity.

In various embodiments, calcium flux is caused by modulation of GPCR activity or ion channel activity. Exemplary GPCRs include, but are not limited to, an H1 histamine receptor, a gastric inhibitory polypeptide (GIP) receptor, a GLP-1 receptor, a glucagon receptor, an S1P₂ sphingosine 1-phosphate receptor, an EP₁ prostaglandin receptor or an EP₃ prostaglandin receptor. An exemplary ion channel includes transient receptor potential A1 (TRPA1).

In some embodiments, the modified photoproteins comprise a mitochondrial signaling sequence. An exemplary mitochondrial signaling sequence is the COX8 mitochondrial sequence, e.g., set forth in SEQ ID NO:8.

### BRIEF DESCRIPTION OF THE DRAWINGS

FIG. 1 depicts an amino acid sequence alignment of photoproteins Clytin (SEQ ID NO:1), Aequorin (SEQ ID NO:2), Mitrocomin (SEQ ID NO:3) and Obelin (SEQ ID NO:4). The calcium-binding helix-turn-helix (HTH)/EF hand motifs are boxed. Sequence identities are noted by an asterisk (*), and sequence similarities are noted by a colon (:).

FIG. 2 depicts a graph summarizing the results of an exemplary experiment to measure the affinities for calcium of wild-type mitochondrial Aequorin (mt-Aequorin, the amino acid sequence of which is set forth in SEQ ID NO:6), wild-type mitochondrial Clytin (mt-Clytin, the amino acid sequence of which is set forth in SEQ ID NO:5), wild-type mitochondrial obelin (mt-Obelin, the amino acid sequence of which is set forth in SEQ ID NO: 7), modified mitochondrial Clytin (K168D) (mt-Clytin K168D, the amino acid sequence of which is set forth in SEQ ID NO:10), and modified mitochondrial Clytin (K168E) (mt-Clytin K168E, the amino acid sequence of which is set forth in SEQ ID NO:12) using transient co-transfection assays in HEK293T cells. The X-axis represents the calcium concentration and the Y-axis represents the normalized log of the ratio between bioluminescence emitted in the presence of the indicated concentration of calcium and the bioluminescence emitted in the presence of 1.5 mM CaCl₂ (log(L/Lmax)).

FIG. 3 depicts a graph summarizing the results of an exemplary experiment to measure receptor-mediated changes in bioluminescence in U-2OS cells transiently co-transfected with a cDNA encoding the H1 histamine receptor and either wild-type mitochondrial Aequorin (mt-Aequorin, the amino acid sequence of which is set forth in SEQ ID NO:6), wild-type mitochondrial Clytin (mt-Clytin, the amino acid sequence of which is set forth in SEQ ID NO:5), wild-type cytosolic Aequorin (wt-Aequorin, the amino acid sequence of which is set forth in SEQ ID NO:2), wild-type cytosolic Clyin (wt-Clytin, the amino acid sequence of which is set forth in SEQ ID NO:1), modified mitochondrial Clytin (mt-Clytin K168D, the amino acid sequence of which is set forth in SEQ ID NO:10) or modified cytosolic Clytin (K168D, the amino acid sequence of which is set forth in SEQ ID NO:9). The X-axis represents the concentration of histamine applied to the cells and the Y-axis represents the bioluminescence measured in relative luminescence units (RLUs).

FIG. 4 depicts a graph summarizing the results of an exemplary experiment to compare receptor-mediated changes in bioluminescence with the changes in bioluminescence induced by permeabilization of the cellular membrane using Triton X-100 in the presence of 1 mM CaCl₂, in U-2OS cells transiently co-transfected with a cDNA encoding the H1 histamine receptor and either wild-type mitochondrial Aequorin (mt-Aequorin, the amino acid sequence of which is set forth in SEQ ID NO:6), wild-type mitochondrial Clytin (mt-Clytin, the amino acid sequence of which is set forth in SEQ ID NO:5), wild-type cytosolic Aequorin (wt-Aequorin, the amino acid sequence of which is set forth in SEQ ID NO:2), wild-type cytosolic Clyin (wt-Clytin, the amino acid sequence of which is set forth in SEQ ID NO:1), modified mitochondrial Clytin (mt-Clytin K168D, the amino acid sequence of which is set forth in SEQ ID NO:10) or modified cytosolic Clytin (K168D, the amino acid sequence of which is set forth in SEQ ID NO:9). The Y-axis represents the bioluminescence measured in relative luminescence units (RLUs). Specific photoproteins transfected into the cells are indicated on the X-axis.

FIG. 5 depicts a graph summarizing the results of an exemplary experiment to measure receptor-mediated changes in bioluminescence in HEK293T cells transiently co-transfected with cDNAs encoding the GIP (gastric inhibitory polypeptide) receptor, a chimeric promiscuous G protein, and either wild-type mitochondrial Aequorin (mt-Aequorin, the amino acid sequence of which is set forth in SEQ ID NO:6), wild-type mitochondrial Clytin (mt-Clytin, the amino acid sequence of which is set forth in SEQ ID NO:5), wild-type mitochondrial Obelin (mt-Obelin, the amino acid sequence of which is set forth in SEQ ID NO: 7), wild-type cytosolic Aequorin (wt-Aequorin, the amino acid sequence of which is set forth in SEQ ID NO:2), wild-type cytosolic Clyin (wt-Clytin, the amino acid sequence of which is set forth in SEQ ID NO:1), wild-type cytosolic Obelin (wt-Obelin, the amino acid sequence of which is set forth in SEQ ID NO: 4), modified mitochondrial Clytin (mt-Clytin K168D, the amino acid sequence of which is set forth in SEQ ID NO:10) or modified cytosolic Clytin (Clytin K168D, the amino acid sequence of which is set forth in SEQ ID NO:9). The X-axis represents the concentration of GIP applied to the cells and the Y-axis represents the bioluminescence measured in relative luminescence units (RLUs).

FIGS. 6A-6E depict graphs summarizing the results of exemplary experiments to measure receptor-mediated changes in bioluminescence in HEK293T cells transiently co-transfected with cDNAs encoding the GLP-1 (glucagon-like peptide-1) Receptor, the Glucagon receptor, the S1P2 (sphingosine 1-phosphate receptor 2) Receptor, the EP 1 receptor and the EP3 receptor (the latter two receptors for prostaglandin E₂) a chimeric promiscuous G protein (for GLP-1 receptor, glucagon Receptor and S1P2 Receptor), and modified cytosolic Clytin (Clytin K168D, the amino acid sequence of which is set forth in SEQ ID NO:9). The X-axis represents the concentration of corresponding ligand for each receptor applied to the cells and the Y-axis represents the bioluminescence measured in relative luminescence units (RLUs).

FIGS. 7A-7F depict graphs summarizing the results of exemplary experiments to measure receptor-mediated changes in bioluminescence in HEK293T cells transiently co-transfected with cDNAs encoding the GIP Receptor (gastrointestinal peptide receptor), the CXCR1 Receptor, the CXCR4 Receptor, the Glucagon Receptor, a GLP-1 Receptor, or the EP1 Receptor; a chimeric promiscuous G protein, and modified cytosolic Clytin (Clytin K168D, the amino acid sequence of which is set forth in SEQ ID NO:9). FIG. 7G depicts a graph summarizing the results of exemplary experiments to measure receptor-mediated changes in bioluminescence in CHO—K1 cells stably expressing the D2 Receptor, a promiscuous G protein, and a modified version of cytosolic Clytin (Clytin K168E, the amino acid sequence of which is set forth in SEQ ID NO:11). The bioluminescence data depicted in the graphs was obtained on a FLIPRTetra Plus high throughput luminescent plate reader (Molecular Devices).

FIG. 8 depicts a graph summarizing the results of an exemplary experiment to measure receptor-mediated changes in bioluminescence in HEK293 cells stably expressing the cDNA encoding TRPA1 cation channel and transiently co-transfected with cDNAs encoding either wild-type mitochondrial Aequorin (mt-Aequorin, the amino acid sequence of which is set forth in SEQ ID NO:6), wild-type mitochondrial Clytin (mt-Clytin, the amino acid sequence of which is set forth in SEQ ID NO:5), wild-type cytosolic Aequorin (wt-Aequorin, the amino acid sequence of which is set forth in SEQ ID NO:2), wild-type cytosolic Clyin (wt-Clytin, the amino acid sequence of which is set forth in SEQ ID NO:1), modified mitochondrial Clytin (mt-Clytin K168D, the amino acid sequence of which is set forth in SEQ ID NO:10) or modified cytosolic Clytin (Clytin K168D, the amino acid sequence of which is set forth in SEQ ID NO:9). The X-axis represents the concentration of allyl isothiocyanate (AITC) applied to the cells and the Y-axis represents the bioluminescence measured in relative luminescence units (RLUs).

### DETAILED DESCRIPTION OF THE INVENTION

The present invention provides modified photoproteins, e.g., modified Clytin, having increased affinity for calcium as well as enhanced bioluminescence relative to wt-Clytin and/or wt-Aequorin and/or wt-Obelin, and their uses as calcium indicators in reporter gene systems and in cell-based assays.

### I. Definitions

In order that the present disclosure may be more readily understood, certain terms are first defined. Additional definitions are set forth throughout the detailed description.

The term “photoprotein” or “Ca²⁺ activated photoprotein,” as used interchangeably herein, refers to a protein which emits light upon binding to calcium. Photoproteins are generally isolated from marine coelenterates and emit visible light in the presence of calcium through an intramolecular reaction. The calcium binding sites of the known photoproteins are similar to those found in other Ca²⁺ binding proteins such as, for example, Calmodulin, however, differ from other Ca²⁺ proteins by a relatively high content of cysteine, histidine, tryptophan, proline and tyrosine residues.

Exemplary photoproteins include, but are not limited to, Obelin, Clytin, Aequorin, Halistaurin, Mnemiospin and Berovin and generally do not include Luciferases. All these photoproteins are complexes of an apoprotein, an imidazopyrazine chromophore (coelenterazine) and oxygen.

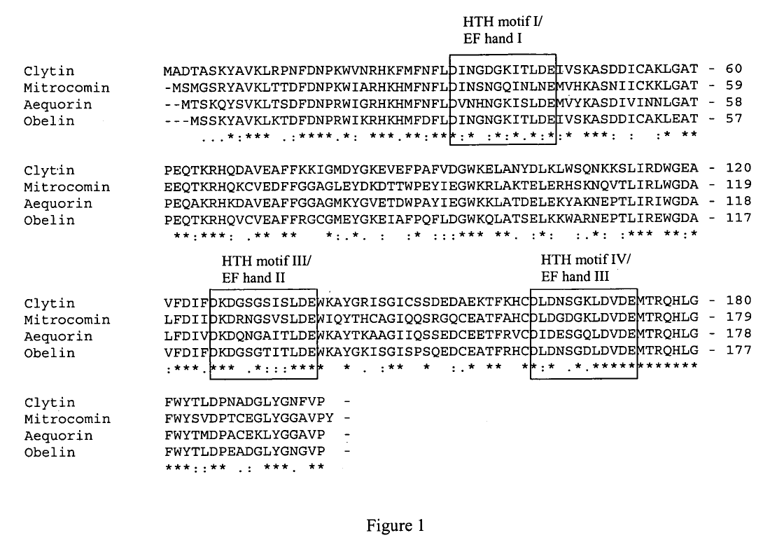

In some embodiments, the present invention provides modified photoproteins. In a particular embodiment, the present invention relates to modified Clytin. Amino acid sequence alignment of photoproteins Clytin (SEQ ID NO:1), Aequorin (SEQ ID NO:2), Mitrocomin (SEQ ID NO:3) and Obelin (SEQ ID NO:4) are depicted in FIG. 1.

The term “modified photoprotein” or “Ca²⁺ activated modified photoprotein,” as used interchangeably herein, refers to an amino acid sequence variant of a wild-type photoprotein (e.g., a variant of wt-Clytin, the amino acid sequence of wt-Clytin is set forth in SEQ ID NO:1), which exhibits an increased affinity for intracellular calcium and enhanced bioluminescence relative to that exhibited by wt-Clytin. In some embodiments, a modified photoprotein according to the present invention includes at least one amino acid modification in an helix-turn-helix (HTH) domain of wt-Clytin (e.g., EF hand III domain), where the modified photoprotein exhibits an increased affinity for intracellular calcium and enhanced bioluminescence relative to that exhibited by wt-Clytin. In some embodiments, a modified photoprotein comprises an amino acid sequence including at least the lysine residue at position 168 of SEQ ID NO:1 replaced with an amino acid other than a histidine, an arginine and a lysine, where the modified photoprotein exhibits an increased affinity for intracellular calcium and enhanced bioluminescence relative to a photoprotein comprising the amino acid sequence set forth in SEQ ID NO:1. In some embodiments, a modified photoprotein comprises an amino acid sequence including at least the lysine residue at position 168 of SEQ ID NO:1 replaced with an aspartic acid, where the photoprotein exhibits an increased affinity for intracellular calcium and enhanced bioluminescence relative to both a photoprotein comprising the amino acid sequence set forth in SEQ ID NO:1 and a photoprotein comprising the amino acid sequence set forth in SEQ ID NO:2.

In exemplary embodiments, a modified photoprotein according to the present invention comprises an amino acid sequence selected from the group consisting of SEQ ID NO:9 (K168D), SEQ ID NO:11 (K168E), SEQ ID NO:15 (K168G), SEQ ID NO:17 (K168N), SEQ ID NO:19 (K168Q), SEQ ID NO:21 (K168S), SEQ ID NO:23 (K168T), SEQ ID NO:25 (K168V) and SEQ ID NO:27 (K168Y). In some embodiments, modified photoproteins of the present invention further include a mitochondrial targeting signal sequence, e.g., a COX8 mitochondrial tag, the amino acid sequence of which is set forth in SEQ ID NO:8.

An EF hand domain is a type of a helix-turn-helix (HTH) structural domain found in a large family of calcium-binding proteins including the photoproteins encompassed by the present invention. It consists of two alpha helices positioned roughly perpendicular to one another and linked by a short loop region (usually about 12 amino acids) that usually binds calcium ions. The EF hand domain takes its name from traditional nomenclature used in describing the protein Parvalbumin, which contains three such motifs and is thought to be involved in muscle relaxation via its calcium-binding activity. EF hand domains also appear in each structural domain of the signaling protein Calmodulin and in the muscle protein Troponin-C.

The term “HTH IV domain” or “EF hand III domain,” refers to the fourth of four helix-turn-helix domains (three of which are EF hand domains) and third of the three EF hand domains found in Clytin, which comprises amino acid residues 162 through 173 and includes the amino acid sequence DLDNSGKLDVDE (SEQ ID NO:31). The HTH IV domain (or the EF hand III domain) of Clytin reportedly binds to calcium at physiologically relevant concentrations.

Without wishing to be bound by theory, it is understood that amino acid sequence variants encompassed by the present invention may differ from the parent amino acid sequence from which they are derived, in the substitution, deletion and/or insertion of one or more amino acids anywhere within the parent amino acid sequence and including at least one amino acid residue substitution in at least one HTH domain (e.g., position 168 of Clytin which resides in the EF hand III domain), where the variants exhibit an increased affinity for calcium (e.g., an EC50 value of 500 nM or lower for intracellular calcium in HEK293T cells) and enhanced bioluminescence. In some embodiments, amino acid sequence variants will possess at least about 70%, or at least about 80%, or at least about 85%, or at least about 90%, or at least about 95%, or at least about 96%, or at least about 97%, or at least about 98%, or at least about 99% identity with the parent sequence (i.e., wt-Clytin set forth in SEQ ID NO:1), where such variants exhibit increased affinity for intracellular calcium and enhanced bioluminescence, and where such variants do not comprise the amino acid sequence set forth in SEQ ID NO:2 or variants thereof.

The term “sequence identity” means that two nucleotide or amino acid sequences, when optimally aligned, such as by the programs GAP or BESTFIT using default gap weights, share at least 70% sequence identity, or at least 80% sequence identity, or at least 85% sequence identity, or at least 90% sequence identity, or at 95% sequence identity or more (e.g., 99% sequence identity or more). For sequence comparison, typically one sequence acts as a reference sequence (e.g., parent sequence), to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI interne server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Software for performing multiple sequence alignments with the MUSCLE (Multiple Sequence Comparison by Log Expectation) algorithm (Edgar, Nucl. Acids Res. 32: 1792 (2004)) is publicly available through the European Molecular Biology Laboratories via the European Bioinformatics Institute interne server.

In one embodiment, a modified photoprotein according to the present invention is based on the amino acid sequence of wt-Clytin (i.e., set forth in SEQ ID NO:1), where the modified photoprotein includes at least the lysine amino acid residue at position 168 replaced with an amino acid residue other than a histidine, an arginine and a lysine. In some embodiments, the lysine at position 168 of SEQ ID NO:1 is replaced with an amino acid selected from aspartic acid, glutamic acid, glycine, asparagine, serine, threonine, valine, tyrosine and glutamine, where the modified photoprotein exhibits an increased affinity for calcium (e.g., an EC50 value of 500 nM or lower in HEK293T cells) and enhanced bioluminescence relative to a wild type photoprotein (e.g., wt-Clytin and/or wt-Aequorin and/or Obelin). In a particular embodiment, a modified photoprotein according to the invention comprises an amino acid sequence comprising at least the lysine at position 168 replaced with an aspartic acid, where the modified photoprotein exhibits an increased affinity for intracellular calcium and enhanced bioluminescence relative to that exhibited by wt-Clytin and wt-Aequorin.

In some embodiments, a modified photoprotein according to the present invention comprises a mitochondrial targeting sequence (e.g., that set forth in SEQ ID NO: 8). In some embodiments, variants of Clytin including a mitochondrial targeting sequence are set forth in SEQ ID NO:10, SEQ ID NO:12, SEQ ID NO:16, SEQ ID NO:18; SEQ ID NO:20, SEQ ID NO:22, SEQ ID NO:24, SEQ ID NO:26 and SEQ ID NO:28.

Suitable amino acids to replace lysine at position 168 of SEQ ID NO:1 include any naturally-occurring amino acid other than a histidine, an arginine and a lysine. In some embodiments, suitable amino acids to replace lysine at position 168 include one of a naturally occurring amino acid selected from an aspartic acid, a glutamic acid, an asparagine, a glycine, a serine, a threonine, a valine, a tyrosine and a glutamine. Non-naturally occurring amino acids and amino acid derivatives which are well known in the art could also be used to replace the lysine at position 168.

The term “bioluminescence,” “luminescence,” “bioluminescent” or “luminescent,” as used herein, relates to the ability of a modified photoprotein of the present invention to emit visible light upon binding to a divalent cation such as, for example, Ca²⁺. Bioluminescence reactions typically require three major components: a luciferin, a luciferase and molecular oxygen. However other components may also be required, including cations (e.g., Ca²⁺ and Mg²⁺) and cofactors (e.g., ATP, NAD(P)H). Luciferases are enzymes that catalyze the oxidation of a substrate, luciferin, and produce an unstable intermediate. Light is emitted when the unstable intermediate decays to its ground state, generating oxyluciferin. Bioluminescence can be measured using one or more techniques known in the art and those described herein, including, but not limited to, use of luminometers such as, e.g., Victor2 and Lumilux (PERKINELMER), FLIPR and FlexStation (MOLECULAR DEVICES/MDS ANALYTICAL), Mithras (BERTHOLD TECHNOLOGIES), FDSS (HAMAMATSU PHOTONICS) and PHERAstar (BMG LABTECH).

The term “enhanced bioluminescence,” as used herein, refers to any increase in bioluminescence of a modified photoprotein relative to a wt-photoprotein in the presence of Ca²⁺. For example, in an exemplary embodiment, bioluminescence of a modified photoprotein such as a modified Clyin (e.g., Clytin having an amino acid modification at position 168) described herein is enhanced relative to the bioluminescence of wt-Clytin and/or wt-Aequorin, as measured either in living cells stimulated with a substance that increases intracellular Ca²⁺, or in permeabilized cells exposed to solutions containing varying concentrations of Ca²⁺. The bioluminescence of a modified photoprotein in the presence of calcium may be increased by about 1.5%, or about 2%, or about 3%, or about 4%, or about 5%, or about 10%, or about 20%, or about 30%, or about 40%, or about 50%, or about 60%, or about 70%, or about 80%, or about 90%, or greater than 90%, relative to that of a wt-photoprotein (e.g., wt-Clytin and/or wt-Aequorin and/or Obelin). In some embodiments, the bioluminescence of a modified photoprotein in the presence of calcium is increased by about 1.5-fold, or 2-fold, or 5-fold, or 10-fold, or 15-fold, or 20-fold, or 25-fold, or 30-fold, or 35-fold, or 40-fold, of 45-fold, or 50-fold, or 55-fold, or 60-fold, or 65-fold, or 70-fold, or 75-fold, or 80-fold, or 85-fold, of 90-fold, or greater than 90-fold, relative to a wt-photoprotein (e.g., wt-Clytin and/or wt-Aequorin).

It is well known that intracellular calcium acts as a modulator of many important physiological responses and pathophysiological conditions. In most of these instances, extracellular signals are received through receptors (e.g., GPCRs and ion channels) and converted to changes in intracellular Ca²⁺ concentration, which results in Ca²⁺ sensitive changes inside the cell, including but not limited to, modulation of Ca²⁺ sensitive kinases, proteases and transcription factors. Accordingly, measurement of intracellular Ca²⁺ concentration is essential in understanding intracellular processes and modulation of cellular proteins. Furthermore, the central role of Ca²⁺ in intracellular signaling makes it a very attractive reporter in drug discovery. Many drug target classes important for the pharmaceutical industry including, but not limited to, G-protein-coupled receptors (GPCRs), ion channels and transporters trigger a Ca²⁺ mobilization upon activation, referred to as “calcium flux.”

Changes in intracellular Ca²⁺ concentration or calcium flux can be detected using fluorescent dyes (e.g., fura-2 and indo-1) (See, e.g., R. Y. Tsien, Nature 290, 527 (1981); R. Y. Tsien, T. Pozzan, T. J. Rink, J. Cell. Biol. 94, 325 (1982)), the Ca²⁺ sensitive bioluminescent jellyfish protein, Aequorin, (e.g., E. B. Ridgway and C. C. Ashley, Biochem. Biophys. Res. Commun. 29, 229 (1967)) or Ca²⁺ sensitive microelectrodes (e.g., C. C. Ashley and A. K. Campbell, Eds., Detection and Measurement of Free Ca²⁺ in cells (Elsevier, North-Holland, Amsterdam, 1979)). In an exemplary experiment, intracellular calcium concentration can be measured by adding coelenterazine cofactor to mammalian cells expressing a photoprotein and detecting photon emission, which is indicative of intracellular calcium concentration.

The present invention provides modified photoproteins which exhibit an increased affinity for intracellular Ca²⁺ relative to the known photoproteins. Accordingly, the modified photoproteins of the present invention are more sensitive to changes in intracellular Ca²⁺ concentration and, accordingly, are superior to the known proteins and reagents for detecting calcium flux. Because the modified photoproteins exhibit a greater sensitivity to changes in intracellular calcium concentration than wild-type photoproteins, the modified photoproteins are extremely valuable to use in assays for screening for modulators of GPCR or ion channel activity, especially screening for modulators which might result only in a small change in intracellular calcium concentration.

The term “increased affinity for intracellular calcium,” as used herein, refers to any increase in the affinity of a modified photoprotein according to the present invention (e.g., Clytin set forth in SEQ ID NO:1 having an amino acid substitution at position 168) for intracellular calcium relative to a wild-type photoprotein (e.g., wt-Aequorin and/or wt-Clytin and/or wt-Obelin). For example, affinity for intracellular calcium may be increased by about 1.5%, or about 2%, or about 3%, or about 3.5%, or about 4%, or about 5%, or about 10%, or about 20%, or about 30%, or about 40%, or about 50%, or about 60%, or about 70%, or about 80%, or about 90%, or about 95%, or about 96%, or about 97%, or about 98%, or about 99% or more, relative to a wild-type photoprotein (e.g., wt-Clytin and/or wt-Aequorin and wt-Obelin). In some embodiments, increased affinity for intracellular calcium refers to a decrease in the EC50 value of a modified photoprotein for intracellular calcium relative to a wild-type photoprotein (e.g., wt-Clytin and/or wt-Aequorin and/or wt-Obelin). Affinity of a photoprotein for calcium can be measured using well known techniques and assays in the art, including but not limited to, the ones described herein. In an exemplary assay, described in the Examples herein, calcium affinity is measured by loading photoprotein-expressing cells with coelenterazine followed by measurement of bioluminescence emitted by the cells upon the addition of varying concentrations of calcium to the cells.

The term “EC50 value for intracellular calcium,” as used herein, refers to the concentration of free calcium that elicits a luminescent signal (i.e., bioluminescence) to a level which is 50% of the signal observed for the luminescent signal in the presence of a saturating amount of calcium (i.e., a concentration of calcium above which further increases in calcium concentration do not produce further increases in luminescent signal). The EC50 value for intracellular calcium, as used herein, is a measure of the affinity of a modified photoprotein for intracellular calcium. The EC50 value can be measured using one or more assays known in the art and those described herein, e.g., in the Examples section infra. In an exemplary embodiment, a modified photoprotein according to the invention has an EC50 value of 500 nM or lower for intracellular calcium in HEK293T cells, where the modified photoprotein is not wt-Aequorin or a variant thereof (e.g., does not comprise an amino acid sequence set forth in SEQ ID NO:2, or a variant thereof).

In some embodiments, the EC50 value for intracellular calcium of a modified photoprotein is decreased by about 1.5%, or 2%, or 3%, or 4%, or 5%, or 10%, or 15%, or 20%, or 25%, or 30%, or 50%, or 60%, or 70%, or 80%, or 90%, or 95%, or greater than 95%, relative to the EC50 value of a wild-type photoprotein (e.g., wt-Clytin and/or wt-Aequorin and/or wt-Obelin). In some embodiments, the EC50 value of a modified photoprotein of the present invention is decreased by about 10 nM, or 20 nM, or nM, or 40 nM, or 50 nM, or 60 nM, or 70 nM, or 80 nM, or 90 nM, or 100 nM, or 110 nM, or 120 nM, or 130 nM, or 140 nM, or 150 nM, or 160 nM, or 170 nM, or 180 nM, or 190 nM, or 200 nM, or 210 nM, or 220 nM, or 230 nM, or 240 nM, or 250 nM, or 260 nM, or 270 nM, or 280 nM, or 290 nM, or 300 nM, or 310 nM, or 320 nM, or 330 nM, or 340 nM, or 350 nM, or 360 nM, or 370 nM, or 380 nM, or 390 nM, or 400 nM, or 410 nM, or 420 nM, or 430 nM, or 440 nM, or 450 nM, or decreased by more than 450 nM, relative to the EC50 value of a wild-type photoprotein (e.g., wt-Clytin and/or wt-Aequorin and/or wt-Obelin).

The term “GPCR” refers to G-protein coupled receptors, which are involved in various cellular signal transduction pathways. As one of the largest and most diverse protein families in nature, the G-protein coupled receptor (GPCR) superfamily plays important roles in a variety of biological and pathological processes such as development and proliferation, neuromodulation, angiogenesis, metabolic disorders, inflammation, and viral infection. It is one of the most targeted protein families in pharmaceutical research today. All members of the GPCR superfamily share a similar seven transmembrane domain, however, can be grouped into classes on the basis of shared sequence motifs. For example, Class A includes Rhodopsin-like GPCRs, Class B includes Secretin-like GPCRs, Class C includes Metabotropic glutamate/pheromone GPCRs, Class D includes fungal pheromone GPCRs, and Class E includes cAMP GPCRs. Additional GPCRs can be classified as Frizzled/Smoothened GPCRs, Vomeronasal GPCRs and some that remain unclassified.

Table I below provides an enumeration of the nucleic acid and amino acid sequences discussed herein along with the corresponding Sequence Identifiers (SEQ ID NOs).

TABLE I

| SEQ ID NO | Brief Description |
|---|---|
| 1 | Wild type Clytin (wt-Clytin) amino acid sequence |
| 2 | Wild-type Aequorin (wt-Aequorin) amino acid sequence |
| 3 | Wild-type Mitrocomin (wt-Mitrocomin) amino acid sequence |
| 4 | Wild-type Obelin (wt-Obelin) amino acid sequence |
| 5 | Mitochondrial Wild-type Clytin (mt-Clytin) amino acid sequence |
| 6 | Mitochondrial Wild-type Aequorin (mt-Aequorin) amino acid sequence |
| 7 | Mitochondrial Wild-type Obelin (mt-Obelin) amino acid sequence |
| 8 | COX 8 mitochondrial tag amino acid sequence |
| 9 | K168D modified Clytin amino acid sequence |
| 10 | Mitochondrial K168D modified Clytin amino acid sequence (mt-Clytin |
| | K168D) |
| 11 | K168E modified Clytin amino acid sequence |
| 12 | Mitochondrial K168E modified Clytin amino acid sequence (mt-Clytin |
| | K168E) |
| 13 | K168H modified Clytin amino acid sequence |
| 14 | Mitochondrial K168H modified Clytin amino acid sequence (mt-Clytin |
| | K168H) |
| 15 | K168G modified Clytin amino acid sequence |
| 16 | Mitochondrial K168G modified Clytin amino acid sequence (mt-Clytin |
| | K168G) |
| 17 | K168N modified Clytin amino acid sequence |
| 18 | Mitochondrial K168N modified Clytin amino acid sequence (mt-Clytin |
| | K168N) |
| 19 | K168Q modified Clytin amino acid sequence |
| 20 | Mitochondrial K168Q modified Clytin amino acid sequence (mt-Clytin |
| | K168Q) |
| 21 | K168S modified Clytin amino acid sequence |
| 22 | Mitochondrial K168S modified Clytin amino acid sequence (mt-Clytin |
| | K168S) |
| 23 | K168T modified Clytin amino acid sequence |
| 24 | Mitochondrial K168T modified Clytin amino acid sequence (mt-Clytin |
| | K168T) |
| 25 | K168V modified Clytin amino acid sequence |
| 26 | Mitochondrial K168V modified Clytin amino acid sequence (mt-Clytin |
| | K168V) |
| 27 | K168Y modified Clytin amino acid sequence |
| 28 | Mitochondrial K168Y modified Clytin amino acid sequence (mt-Clytin |
| | K168Y) |
| 29 | K168R modified Clytin amino acid sequence |
| 30 | Mitochondrial K168R modified Clytin amino acid sequence (mt-Clytin |
| | K168R) |
| 31 | Wild-type Clytin EF hand III domain amino acid sequence |
| 32 | Wild type Clytin (wt-Clytin) nucleic acid sequence |
| 33 | Wild-type Aequorin (wt-Aequorin) nucleic acid sequence |
| 34 | K168D modified Clytin nucleic acid sequence |
| 35 | Mitochondrial Wild-type Clytin (mt-Clytin) nucleic acid sequence |
| 36 | Mitochondrial Wild-type Aequorin (mt-Aequorin) nucleic acid |
| | sequence |
| 37 | Mitochondrial K168D modified Clytin (mt-Clytin K168D) nucleic acid |
| | sequence |
| 38 | K168D sense primer |
| 39 | K168D antisense primer |
| 40 | K168E sense primer |
| 41 | K168E antisense primer |
| 42 | K168G sense primer |
| 43 | K168G antisense primer |
| 44 | K168N sense primer |
| 45 | K168N antisense primer |
| 46 | K168Q sense primer |
| 47 | K168Q antisense primer |
| 48 | K168V sense primer |
| 49 | K168V antisense primer |
| 50 | K168S sense primer |
| 51 | K168S antisense primer |
| 52 | K168T sense primer |
| 53 | K168T antisense primer |
| 54 | K168Y sense primer |
| 55 | K168Y antisense primer |
| 56 | K168R sense primer |
| 57 | K168R antisense primer |
| 58 | K168H sense primer |
| 59 | K168H antisense primer |
| 60 | H1 forward PCR primer |
| 61 | H1 reverse PCR primer |

### II. Exemplary Photoproteins

The present invention relates to modified Ca²⁺ binding photoproteins. The Ca²⁺ binding photoproteins are protein-substrate-oxygen complexes used by bioluminescent organisms in the phyla Protozoa, Cnidaria and Ctenophora to produce light. Exemplary Ca²⁺ binding photoproteins that have been described in the art include Thalassicolin, Aequorin, Mitrocomin, Clytin (also known as Phialidin), Obelin, Mnemiopsin and Berovin. Four of the photoproteins, Aequorin, Mitrocomin, Clytin and Obelin, are from class Hydrozoa, phylum Cnidaria and are relatively small in size (21.4-27.5 kDa).

Since coelenterazine is the common luminescent chromophore contained in the photoproteins Aequorin, Mitrocomin, Clytin and Obelin, the light-emitting reaction is thought to be the same in these four photoproteins (Tsuji et al., Photochem. Photobiol., 62: 657-661 (1995)). Conventional nomenclature defines these photoprotein designations as the polypeptide complexed with the chromophore, whereas the photoprotein lacking the chromophore is designated an apoprotein (e.g., apoaequorin, apoclytin, apoobelin and apomitrocomin). In addition, the Ca²⁺ binding photoproteins appear to retain a tightly bound O₂ molecule. Upon binding of calcium ions to the photoproteins, the protein catalyzes oxidation of coelenterazine and O₂ to coelenteramide, CO₂, and photons with emission maxima at 470 nm.

Due to its low toxicity, Aequorin has been used as an intracellular calcium indicator since the early 1960s. Original use of Aequorin employed biochemically purified enzyme microinjected into the cells of interest (Blinks et al. Pharmacol Rev., 28: 1-93 (1976)).

Molecular cloning of apoaequorin indicated that it consists of 189 amino acid residues in a single polypeptide chain and contains 4 HTH domains, 3 of which have amino acid sequences characteristic of EF hand Ca²⁺ binding sites (Inouye et al., Proc. Natl. Acad Sci USA, 82: 3154-3158 (1985)). Cloning of the gene for Aequorin opened the door for recombinant expression in cells or even entire organisms. Expression of recombinant aequorin cDNA tagged with subcellular targeting signal sequences transports Aequorin to specific subcellular compartments, thereby permitting measurement of calcium concentrations specifically within those compartments (Rizzuto et al., Methods Cell Biol. 40: 339-358 (1994)). Such studies with Aequorin selectively targeted to mitochondria have revealed that when Ca²⁺ is released from endoplasmic reticulum (ER) stores, mitochondria accumulate calcium to concentrations exceeding those in the cytosol.

Many GPCRs signal by stimulating calcium release from ER stores via inositol triphosphate. Accordingly, Aequorin has been utilized to measure GPCR-mediated calcium flux. The ease of measuring intracellular calcium flux with mitochondrially targeted Aequorin has permitted development of high throughput assays for modulators of a wide variety of GPCRs (Stables et al., Anal. Biochem., 252: 115-126 (1997); Ungrin et al., Anal. Biochem., 272: 34-42 (1999)). In addition, Aequorin has been utilized for high throughput analysis of calcium ion channel function (Walstab et al., Anal. Biochem., 368: 185-192 (2007)).

### III. Structure of Clytin

Clytin, another photoprotein also known as Phialidin, was cloned from the hydroid Clytia gregarium (formerly known as Phialidium gregarium).

The cloning and sequence analysis of the cDNA for Ca²⁺ activated Clytin (also referred to as Clytin-I) was first described by Inouye et al. (FEBS, 315, 343-346 (1993)). Clytin consists of 189 amino acid residues with about 64% amino acid sequence identity with Aequorin, and includes 4 HTH domains, 3 of which are EF-hand domains that bind Ca²⁺. The amino acid sequence of wild-type Clytin is set forth in SEQ ID NO:1. Regeneration of purified apoclytin with colenterazine, O₂, 2-mercaptoethanol and EDTA leads to Clytin, just as in case of regeneration of apoaequorin into Aequorin. The bioluminescence reaction for Clytin is thought to be similar to that of Aequorin, as it has been reported that addition of calcium to the reconstituted Clytin results in the emission of light with a wavelength maximum of 470 nm (Inouye and Sahara, Protein Expr. Purif., 53: 384-389 (2007)). However, Clytin displays a lower affinity for calcium than Aequorin. A second isotype of Clytin, termed Clytin-II, has been recently cloned from Clytia gregarium (Inouye, J. Biochem., 143: 711-717 (2008)). Clytin-I and Clytin-II share 88.4% amino acid identity, a similar affinity for calcium, and similar total quantum yield with respect to luminescence, however, they differ kinetically with Clytin-II displaying a 4.5-fold higher peak luminescence than both Clytin-I and Aequorin.

### IV. Generation of Modified Photoproteins

The modified photoproteins of the present invention can be made using any suitable methods known in the art. For example, standard techniques for site-directed mutagenesis of nucleic acids may be used such as those described, for example, in the laboratory manual entitled Molecular Cloning by Sambrook, Fritsch and Maniatis. Additionally, standard molecular biology techniques involving polymerase chain reaction (PCR) mutagenesis may be used.

In some embodiments, the modified photoproteins are generated using standard genetic engineering techniques. For example, a nucleic acid molecule encoding a wt-photoprotein or a portion thereof can be cloned into a suitable vector for expression in an appropriate host cell. Suitable expression vectors are well known in the art and typically include the necessary elements for the transcription and translation of the modified photoprotein coding sequence.

Modified photoproteins described herein may also be synthesized chemically from amino acid precursors using methods well known in the art, including solid phase peptide synthetic methods.

Expression of modified photoproteins can be accomplished in cells from eukaryotic hosts such as yeasts, insects or mammals, or in prokaryotic host cells, e.g., bacteria such as E. coli.

In some embodiments, modified photoproteins include a signal sequence for targeting such photoproteins to a particular compartment within a cell, for example, in order to detect calcium flux in a particular cellular compartment. In a particular embodiment, the modified photoproteins are specifically targeted to the mitochondria, e.g., by including a mitochondrial signaling sequence (e.g., COX8 signal sequence as described herein) at the amino-terminus of the modified protein.

In some embodiments, modified photoproteins include a tag or fusion, either at the N-terminus, C-terminus, or internal region, for detection and/or purification of the modified photoprotein. Such sequence tags include, but are not limited to, hemagglutinin (HA) tag, FLAG tag, myc tag, hexahistidine tag (SEQ ID NO: 62) and glutathione S-transferase (GST) fusion.

In some embodiments, modified photoproteins may be expressed on the surface of a bacteriophage such that each phage contains a DNA sequence that codes for an individual modified photoprotein displayed on the phage surface. In this approach, a library of modified photoproteins are made by synthesizing random or semi random oligonucleotides at selected positions in a photoprotein sequence chosen to generate a variety of amino acids at these positions. The encoding DNA is inserted into an appropriate phage vector, packaged into a phage particle and used to infect a suitable bacterial host. Each of the sequences is thus cloned in one phage vector and the modified photoprotein of interest (e.g., having a mutation at position 168 in case of clytin and having increased affinity for Ca2+) can be isolated and the nucleotide sequence encoding selected modified photoproteins determined by nucleotide sequencing.

### V. Transfecting a Nucleic Acid Molecule Encoding a Modified Photoprotein into a Suitable Cell

A variety of methods known to those skilled in the art are available for transfecting nucleic acid molecules into suitable cells. For example, calcium phosphate, cationic lipids, and cationic polymers (such as polyethyleneimine) may be complexed with nucleic acid molecules and applied to cells, which subsequently internalize the complex and transcribe and/or translate the nucleic acid molecules. Alternatively, electrophysical methods such as electroporation, biolistic gene transfer, and microinjection may be used to generate transient openings within the cellular plasma membrane to allow diffusion of nucleic acid molecules across the plasma membrane. Viral vectors such as lentivirus, baculovirus, adenovirus, and adeno-associated virus may also be engineered to include a recombinant gene, and infection of the recombinant virus to receptive cells results in expression of the encoded recombinant protein. In theory, any mammalian cell line may be transfected with a nucleic acid molecule encoding a modified photoprotein encompassed by the present invention. Exemplary cell lines include, but are not limited to, CHO, COS, HEK293, U-2OS, HeLa, and NIH3T3. Such transfected cell lines may be used in assays, e.g., 24 to 72 hours following transfection. Alternatively, if the nucleic acid molecule of interest has been inserted into a plasmid also containing a gene for a selectable marker, the cells may be subjected to selection with a compound that is toxic to untransfected cells, but that is inactivated by the coexpressed selectable marker. Exemplary selection agents include geneticin, hygromycin, zeocin, and puromycin.

### VI. Measuring the Affinity of the Modified Photoprotein for Intracellular Calcium

The affinity of a calcium-activated photoprotein for intracellular calcium may be determined by several methods known to those skilled in the art, including those described herein. Generally, each method involves the use of solutions containing defined concentrations of calcium in the presence of calcium-binding buffers, such as EGTA or EDTA, that have known affinities for calcium. Thus, the effective concentration of free calcium in such buffered solutions can be readily calculated.

The photoproteins may be expressed in bacteria such as E. coli and purified and subsequently complexed with the substrate coelenterazine. The photoproteins may also be expressed in mammalian cells and cellular lysates prepared from the cells and subsequently incubated with coelenterazine. Alternatively, cells expressing the photoproteins may be loaded with coelenterazine, then permeabilized by a detergent such as Triton X-100 or digitonin. In each case, the photoprotein preparation is exposed to the buffered calcium solution in a luminometer designed to quantify the emission of photons generated by the oxidation of coelenterazine.

### VII. Measuring GPCR Mediated or Ion Channel Mediated Bioluminescence with the Modified Photoprotein

Modified photoproteins may be used to measure GPCR activity or ion channel activity in intact, live cells in a bioluminescence assay. In an exemplary experiment, cells are transfected with a nucleic acid molecule encoding a GPCR or an ion channel and another nucleic acid molecule encoding an apophotoprotein. Alternatively, cells expressing endogenous GPCR or endogenous ion channel may be used, which are transfected with a nucleic acid molecule encoding an apophotoprotein. The transfected cells are maintained in media for a period of time to allow expression of the encoded recombinant proteins (typically 24-72 hours). Alternatively, the transfected cells are treated with one or more selective agents, typically for 1-3 weeks, to enrich for cells containing a stably integrated gene or genes.

The live transfected cells are incubated with the chromophore coelenterazine, which can either be in native form or in a chemically modified form. Coelenterazine readily traverses the plasma membranes and enters the cell to complex with the apophotoprotein. In case of GPCRs, cells containing the reconstituted photoprotein are then exposed to a ligand for the GPCR, and luminescence is quantified with a luminometer.

### VIII: Screening Methods for Identifying Modulators of GPCR Activity

The invention also provides methods of screening for modulators, e.g., activators, inhibitors, stimulators, enhancers, agonists, and antagonists, of GPCR activity. For example, the modified photoproteins can be used for identifying modulators of GPCR activity by detecting calcium flux in the presence of a modulator.

Stimulation of cytosolic free calcium concentrations is a primary signal transduction pathway for many GPCRs. Typically, binding of an agonist to certain GPCRs elicits a conformational change that activates heterotrimeric G proteins of the Gq/11 class. The activated GTP-bound form of the alpha subunit of Gq activates the enzyme phospholipase c, which in turn, catalyzes the cleavage of membrane bound lipid phosphatidylinositol. This cleavage reaction generates diacylglycerol, which remains associated with the lipid bilayer, and inositol triphosphate, which is released into the cytosol. Inositol triphosphate binds and activates a calcium channel on the endoplamsic reticulum (ER) to mobilize calcium from stores in the ER into the cytosol. Such GPCR-mediated changes in cytosolic free calcium concentrations (i.e., calcium flux) lead to a number of biologically important downstream responses, including alterations in cellular phosphorylation and transcription. Subsequently, calcium from the cytosol accumulates in mitochondria. Additionally, it has been reported that direct transfer of calcium from the ER to the mitochondria may occur.

Several methods for detecting and quantifying changes in cytosolic and mitochondrial free calcium have been developed. For example, fluorescent dyes have been developed that change either the fluorescence intensity, or the emission or excitation maximum wavelength, upon binding of calcium. Such dyes may be applied to cells to accumulate in the cytosol. Changes in cytosolic calcium concentrations result in changes in fluorescence intensity or wavelength maxima that may be quantified by use of fluorometric detectors. In addition, calcium-activated photoproteins such as Aequorin, Clytin and Obelin may be used to monitor changes in intracellular calcium. Such photoproteins have an advantage in that they may be fused with sequences that direct them to different organelles, such that calcium concentrations in various cellular compartments may be measured.

Due to the simple and sensitive methods available for monitoring changes in calcium concentrations within cellular compartments, screening for modulators of GPCR activity often utilizes such methods. Typically, a cell line expressing an endogenous or recombinant GPCR is loaded with a calcium-sensitive dye such as Fluo-4 in 96- or 384-well plates. A fluorometric plate reader with liquid handling capability simultaneously quantifies fluorescence as the compounds of interest are added to the plate. A second addition of a known agonist may be added in order to determine whether the compound in the first addition is an antagonist and inhibits the activity of the known agonist. Such screens are capable of analyzing nearly 100 plates per day per device, or up to 40,000 compounds. Such high throughput screens have been and currently are commonly used to discover novel compounds interacting with such exemplary GPCRs as histamine receptors (H₁-H₄), 5-HT receptors (5-HT_{1A}, 5-HT_{1B}, 5-HT_{1D}, 5-HT_{2A}, 5-HT_{2B}, 5-HT_{2C}, 5-HT₄, 5-HT₆ and 5-HT₇), dopamine receptors (D₁-D₅), adrenoceptors (α1A, α1B, α1D, α2A, α2B, α2C, β1, β2, β3), glucagon-like peptide receptors (GLP-1 receptor), opioid receptors (δ, κ, μ).

However, screening efforts performed with fluorescent calcium assays have several limitations. For example, intrinsically fluorescent compounds may interfere with the assay, or the signal to background ratio is not sufficient to miniaturize to a 1536 well format, and a relatively high proportion of false positives is observed. Luminescent calcium assays performed with cells expressing the GPCR of interest with Aequorin have been demonstrated to overcome some of these limitations, resulting in enhanced sensitivity and higher throughput (Gilchrist et al., J. Biomol. Screen., 13: 486-493 (2008)).

This invention is further illustrated by the following examples which should not be construed as limiting. The contents of all references, patents and published patent applications cited throughout this application, as well as the Figures, are incorporated herein by reference.

### EXAMPLES

### Example 1

### Generation of Clytin Variants

Versions of Clytin were generated which were either unmodified for cytosolic expression or contained a mitochondrial targeting sequence. In an exemplary experiment, the cDNAs encoding unmodified wild-type clytin (referred to as cyto-clytin-wt) and clytin with a COX8 mitochondrial leader sequence (referred to as mt-clytin-wt) were chemically synthesized by GenScript. The cDNAs were subsequently subcloned into the mammalian expression vector pcDNA3.1, under the control of a CMV promoter. (INVITROGEN). Mutations including a lysine to aspartic acid mutation at position 168 (K168D) were introduced into the clytin cDNAs by site-directed mutagenesis using the QuikChange kit (STRATAGENE).

The codon AAA encoding the lysine at position 168 in Clytin was changed to one of aspartic acid, glutamic acid, asparagine, glycine, glutamine, valine, serine, threonine, tyrosine, arginine and histidine. The primers used for making these modifications are summarized in Table I supra.

### Example 2

### Measurement of Calcium Affinities of Wild-Type Clytin, Modified Clytin K168D and Wild-Type Aequorin

Following the generation of modified forms of Clytin, the various variants were compared with wt-Aequorin for their affinity for intracellular calcium in transient transfection assays using co-transfection with the H1 histamine receptor (Genbank Accession No. NM_{—}000861, obtained by PCR using H1-specific primers from a human brain cDNA library, forward primer 5′-GCCGCCACCATGAGCCTCCCCA ATTCCTC-3′ (SEQ ID NO:60), reverse primer 5′-TCATCAGGAGCGAATATGCAG AATTCTC-3′ (SEQ ID NO:61).

In an exemplary experiment, HEK293T cells (ATCC-CRL-11268) were transiently co-transfected with 2 μgs of each of pcDNA3.1-H1 (containing the cDNA encoding the H1 histamine receptor) and pcDNA3.1 containing the cDNAs encoding wild type mitochondrial Clytin (referred to as mt-Clytin), modified mitochondrial Clytin having a lysine to aspartic acid mutation at position 168 (referred to as mt-Clytin K168D), modified mitochondrial Clytin having a lysine to glutamic acid mutation at position 168 (referred to as mt-Clytin K168E), wild-type mitochondrial Obelin (referred to as mt-Obelin) or wild type mitochondrial Aequorin (referred to as mt-Aequorin) using the Targefect-293 reagent (TARGETING SYSTEMS). 48 hours later, the cells were detached using Accutase (MILLIPORE), centrifuged, and resuspended in 5 coelenterazine in FreeStyle293 media (INVITROGEN), for incubation for 3-4 hours at room temperature in the dark. After incubation, the cells were centrifuged and resuspended in HBSS/HEPES buffer without Ca²⁺ at a density of 1×10⁶ cells/ml. Next, 100 μl/well of cell suspension for each transfection were added by the use of a Victor2 luminometer plate reader (WALLAC, PERKINELMER), to a 96-well plate containing 50 μL/well Triton X-100 in MOPS/KCl buffer containing 10 mM EGTA and a series of Ca²⁺ concentrations, such that free calcium concentrations ranged from 17 nM to 39.6 μM, followed by for measurement of total bioluminescence at 20 seconds.

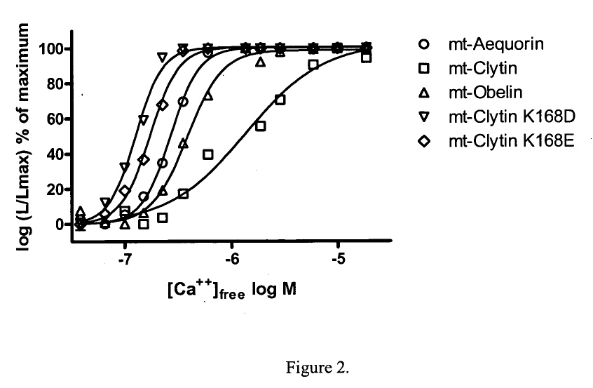

The results of one such experiment are demonstrated in FIG. 2. As shown in FIG. 2, the modified mitochondrial Clytin having a K168D mutation (mt-Clytin K168D) had a very high affinity for Ca²⁺, i.e., an EC50 value of 129 nM. Whereas, the affinities of mt-Clytin and mt-Aequorin were found to be in the range previously reported, i.e., mt-Aequorin exhibited an EC50 value of 269 nM and mt-Clytin exhibited an EC50 value of 1348 nM. The modified mitochondrial Clytin having a K168E mutation also exhibited a relatively high affinity for calcium, i.e., an EC50 value of 173 nM.

### Example 3

### Comparison of GPCR-Mediated Luminescence Exhibited by Various Photoprotein Variants

In another experiment, the ability of the various photoproteins to detect calcium flux in live cells was assessed by adding varying concentrations of histamine to cells in order to activate the H1 histamine receptor followed by measurement of bioluminescence of the various photoproteins. U-2OS cells were transfected with a cDNA encoding H1 histamine receptor along with a cDNA encoding either a wild-type photoprotein or a modified photoprotein according to the invention, as described above in Example 2, except that Lipofectamine-2000 (INVITROGEN) was used as the transfection reagent. On the second day, the cells were trypsinized, counted, and plated in a white tissue-culture treated 96-well plate (COSTAR) at a density of about 50,000 cells/well in growth media consisting of DMEM containing 10% fetal bovine serum, non-essential amino acids, HEPES and penicillin/streptomycin. On the third day, the medium was removed and cells were washed once with 200 μl/well of HBSS/HEPES (containing Ca²⁺). Cells were subsequently incubated with 5 μM coelenterazine in HBSS/HEPES (containing Ca²⁺) in a volume of 200 μl/well, for 3-4 hours at room temperature in the dark. After incubation, the coelenterazine solution was removed and cells were washed and replaced with HBSS/HEPES buffer containing both Ca²⁺ and Mg²⁺. 50 μl/well of histamine at varying concentrations in HBSS/HEPES containing both Ca²⁺ and Mg²⁺ was added to the cells and total bioluminescence was measured at 20 seconds using a Victor2 luminometer.

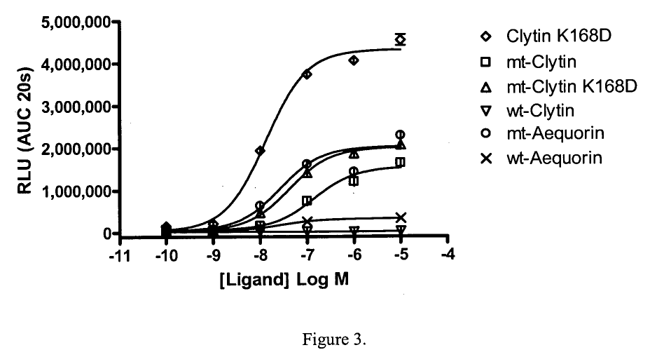

The results of one such exemplary experiment are depicted in the graph in FIG. 3. As shown in the graph, the cytosolic Clytin K169D exhibited a much higher luminescence than the other forms of Clytin and Aequorin tested in the experiment.

### Example 4

### Comparison of GPCR-Mediated Versus Total Luminescence Mediated by Various Photoprotein Variants

In a subsequent experiment, GPCR-mediated bioluminescence versus the total bioluminescence exhibited by various photoproteins was compared, as another measure of the effectiveness of a calcium-activated photoprotein. In an exemplary experiment, U-2OS cells were transfected, plated and loaded with coelenterazine as described in Example 3. Luminescence induced by buffer alone or by 10 μM histamine was measured also as described in Example 3. Additionally, luminescence induced by addition of Triton X-100 in the presence of 1 mM calcium was also determined in order to assess the total amount of active photoprotein detectable using saturating calcium concentrations in cells.

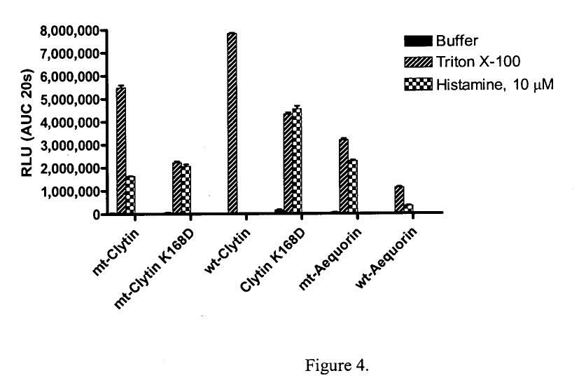

The results of one such experiment are summarized in the bar graph in FIG. 4. As demonstrated in FIG. 4, both the mitochondrial and cytosolic forms of modified Clytin K168D exhibited a GPCR-mediated signal which was close to 100% of the total signal. Whereas, the mitochondrial and cytosolic forms of Aequorin only yielded about 70% and 30% respectively, of the total signal and the mitochondrial and cytosolic forms of wild type Clytin yielded about 30% and 3%, respectively, of the total signal.

### Example 5

### Comparison of Luminescence Exhibited by Various Photoprotein Variants, Induced by a GPCR Coupled to an Exogenous Chimeric G Protein

In another experiment, HEK293T cells were transiently co-transfected with plasmids containing cDNAs encoding the GIP Receptor (Genbank. Accession No. NM_{—}000164; obtained from OPEN BIOSYSTEMS), a promiscuous G protein alpha subunit, and a panel of photoproteins, including mitochondrially targeted wild-type Clytin (wt-Clytin), 11 modified versions of mitochondrially targeted Clytin having an amino acid substitution at position 168 of SEQ ID NO:1 (SEQ ID NOs:10, 12, 14, 16, 18, 20, 22, 24, 26, 28 and 30), cytosolic wild-type Clytin (SEQ ID NO:1), 2 modified versions of cytosolic Clytin having an amino acid substitution at position 168 of SEQ ID NO:1 (SEQ ID NOs:9 and 11), mitochondrially targeted and cytosolic wild-type Aequorin (SEQ ID NOs:6 and 2, respectively), and mitochondrially targeted and Cytosolic wild-type Obelin (SEQ ID NOs: 7 and 4, respectively). The GIP receptor ordinarily couples to the Gs class of G proteins to stimulate the cAMP pathway, and does not couple with Gq to activate calcium flux. However, coexpression of GIP receptor with a chimeric G protein containing sequences from Gαs and Gαq enable the GIP receptor to stimulate calcium flux.

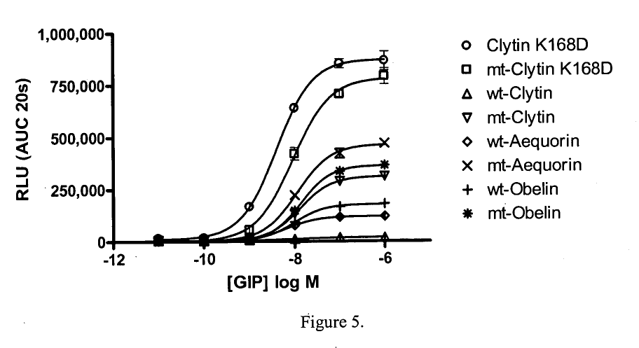

The cells were aliquoted at 100,000 cells/well in a 96-well plate, and subsequently assayed for GPCR mediated bioluminescence, induced by GIP ligand (10⁻¹¹ to 10⁻⁶ M), as well as the total signal determined by addition of 1% Triton X-100 in the presence of 1 mM Ca²⁺. The results of the dose response curve to varying concentrations of GIP with a subset of the photoproteins are summarized in FIG. 5. Cytosolic and mitochondrial Clytin K168D display significantly higher signal intensities than any of the wild-type photoproteins. These data demonstrate that Clytin K168D allows enhanced sensitivity in analysis of GPCRs with non-native coupling to the calcium pathway.

The results obtained with the entire photoprotein panel are summarized below in Table II. The % GPCR/total indicates the signal induced by maximal GIP (1 μM) as a percentage of the signal induced by Triton X-100 in the presence of 1 mM Ca²⁺. Signal to background ratio was calculated as the signal induced by maximal GIP (1 μM) divided by the signal induced by buffer alone. The EC50 value for calcium was determined as described above for FIG. 2 for mitochondrially targeted photoproteins transfected into HEK293T cells in the absence of a cotransfected GPCR or G protein. Affinity for calcium was correlated with the properties of the amino acid side chain at residue 168 of Clytin.

Residues with acidic and hydroxylated side chains, e.g., Clytin K168D, K168T and K168E generally exhibited higher calcium affinity (160 nM, 174 nM and 176 nM, respectively) than wild-type Clytin, Aequorin and Obelin (545 nM, 235 nM and 345 nM, respectively). Two other clytin mutants with hydroxylated or absent side chains, K168S and K168G, exhibited calcium affinities comparable to wild-type Aequorin, however, better than wt-Clytin. Substitutions at position 168 of wt-Clytin, which is a lysine, with hydrophobic amino acid residues (e.g., valine and tyrosine) or carboxamide side chains (e.g., asparagine and glutamine) displayed calcium affinities lower than wt-Aequorin but higher than wild-type Clytin. Substitutions at position 168 of Clytin with basic residues (e.g., histidine and arginine) resulted in calcium affinities comparable to wild-type Clytin.

The EC50 value for receptor-mediated calcium flux induced by the ligand was determined by plotting luminescence (Y-axis) versus ligand concentration applied to the cells (X-axis), and applying a sigmoidal dose-response curve-fitting algorithm (GraphPad Prism). GPCR-mediated signals of the various modified forms of Clytin having an amino acid substitution at position 168 were also related to the nature of the side chain. In addition, the subcellular location of the photoproteins (cytosolic vs mitochondrial) appeared to have an effect on GPCR-mediated signals. In all the mitochondrially targeted version of photoproteins examined, mt-Clytin K168D yielded a lowest EC50 and highest % GPCR/total, highest signal to background ratio, and maximal signal using a GIP ligand. All of the other mitochondrially targeted mutants except for Clytin K168R and wild-type Clytin yielded results that were in the range of those observed for wt-Aequorin. Similarly, among the cytosolic versions of photoproteins examined, Clytin K168D exhibited a lowest EC50 and highest % GPCR/total, highest signal to background ratio and maximal signal using a GIP ligand. Notably, cytosolic Clytin K168D displayed the lowest EC50 and highest maximal signal for a GIP ligand among all of the other photoproteins, cytosolic or mitochondrial.

TABLE II

| | | EC50 for | | | Max signal |
|---|---|---|---|---|---|
| | Calcium | GIP | | Signal: background | with GIP |
| | EC50 (nM) | (nM) | % GPCR/total | with GIP | (RLU × 10⁻⁵) |
| Mt-clytin wt | 545 | 12.9 | 6.8% | 146.5 | 3.14 |
| Mt-clytin | 160 | 9.1 | 34.4% | 188.2 | 7.90 |
| K168D | | | | | |
| Mt-clytin | 176 | 21.6 | 19.8% | 120.8 | 6.07 |
| K168E | | | | | |
| Mt-clytin | 228 | 15.4 | 20.3% | 124.1 | 3.84 |
| K168S | | | | | |
| Mt-clytin | 174 | 13.4 | 22% | 113.4 | 4.26 |
| K168T | | | | | |
| Mt-clytin | 232 | 15.1 | 19.3% | 146.8 | 5.09 |
| K168G | | | | | |
| Mt-clytin | 358 | 21.0 | 10.7% | 115.9 | 3.82 |
| K168N | | | | | |
| Mt-clytin | 335 | 17.4 | 14.2% | 125.8 | 4.78 |
| K168Q | | | | | |
| Mt-clytin | 389 | 16.8 | 18.5% | 158.0 | 3.60 |
| K168V | | | | | |
| Mt-clytin | 284 | 16.4 | 18.5% | 112.8 | 2.89 |
| K168Y | | | | | |
| Mt-clytin | 459 | 19.2 | 14.9% | 148.3 | 4.98 |
| K168H | | | | | |
| Mt-clytin | 581 | 29.1 | 3.3% | 86.8 | 1.89 |
| K168R | | | | | |
| Mt-aequorin | 235 | 11.3 | 23.9% | 84.7 | 4.68 |
| Mt-obelin | 373 | 13.7 | 116% | 172.5 | 3.66 |
| Cyto-clytin | | 9.67 | 0.3% | 7.0 | 0.2 |
| wt | | | | | |
| Cyto-clytin | | 4.01 | 55.6% | 74.3 | 8.76 |
| K168D | | | | | |
| Cyto-clytin | | 7.9 | 26.2% | 66.4 | 6.47 |
| K168E | | | | | |
| Cyto- | | 6.67 | 13.0% | 29.4 | 1.21 |
| aequorin | | | | | |
| Cyto-obelin | | 9.86 | 56.7% | 47 | 1.80 |

### Example 6

### Comparison of Luminescence Exhibited by Clytin K168D induced by GPCRs Selected from Glucagon Receptor, GLP-1 Receptor, S1P2 Receptor, EP 1 Receptor or EP3 Receptor

In another experiment, HEK293T cells were transiently co-transfected with plasmids containing cDNAs encoding an exemplary GPCR selected from a Glucagon Receptor (Genbank Accession No. NM_{—}000160; isolated by RT-PCR from human liver RNA), a GLP-1 Receptor (Genbank Accession No. NM_{—}002062; obtained from CYTOMYX), an S₁P₂ Receptor (GenBank Accession No. NM_{—}004230.3; obtained from OPEN BIOSYSTEMS), an EP₁ Receptor (GenBank Accession No. NM_{—}000995; obtained from OPEN BIOSYSTEMS) or an EP₃ Receptor (GenBank Accession No. NM_{—}198716; obtained from OPEN BIOSYSTEMS), a promiscuous G protein (for Glucagon Receptor, GLP-1 Receptor and S1P₂ Receptor) and a modified version of cytosolic Clytin having an amino acid substitution at position 168 of SEQ ID NO:1 (SEQ ID NO:9).

At 48 h after transfection, the cells were cryopreserved with a CryoMed controlled rate freezer (THERMO SCIENTIFIC), and stored in liquid nitrogen. The cells were subsequently thawed and either directly placed into FreeStyle media (INVITROGEN) containing 5 μM coelenterazine (Thaw/Assay), or plated in a flask overnight in growth media consisting of DMEM/F12 containing 10% fetal bovine serum, non-essential amino acids, and penicillin/streptomycin prior to loading with coelenterazine (Thaw/ON Recovery). After loading with coelenterazine for 3-4 h, the cells were centrifuged and resuspended in HBSS with calcium and magnesium.

The cells were aliquoted at 100,000 cells/well in a 96-well plate, and subsequently assayed for GPCR mediated bioluminescence, induced by a ligand for a Glucagon Receptor (Glucagon at 10⁻⁸ to 10^{-12.5} M), an S₁P₂ Receptor (sphingosine 1-phosphate at 10⁻⁶ to 10^{-11.5} M), an EP₁ Receptor (prostaglandin E₂ at 10⁻⁶ to 10⁻¹¹ M) or an EP₃ Receptor (prostaglandin E₂ at 10⁻⁶ to 10⁻¹¹ M), as well as the total signal determined by addition of 1% Triton X-100 in the presence of 1 mM Ca²⁺.

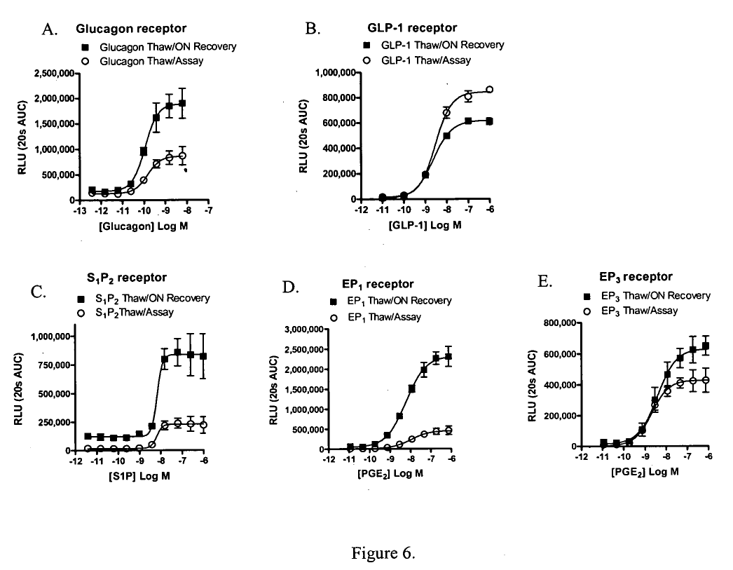

The results are summarized in FIGS. 6A-6E as well as in Table III below. Thaw/Assay indicates dose response of cells that were thawed and directly loaded with coelenterazine prior to assay, and Thaw/ON Recovery indicates dose response of cells allowed to recover in growth media overnight prior to loading and assay. For each receptor except for GLP-1 receptor, cells that were allowed to recover overnight before loading and assay yielded higher signal than cells that were loaded and assayed immediately after thawing. The EC50 values for activation of each receptor by its ligand for each cell treatment method were determined as described above for FIG. 5 for cytosolic clytin K168D photoprotein transfected into HEK293T cells with cotransfected GIP Receptor and G protein. The EC50 values thus calculated are depicted in Table III, and compared with EC50 values determined in fluorescent calcium assays with cell lines stably expressing the indicated receptors (MILLIPORE). The values obtained in luminescent assay were no more than 4-fold greater than in the fluorescent assay. In the cases of Glucagon Receptor and S1P₂, the EC50 values were 6-100-fold lower with the luminescent assay than the fluorescent assay, which indicates that the luminescent assay in some cases exhibits greater sensitivity for detecting calcium flux than traditional fluorescent methods.

TABLE III

| | GLP-1 | Glucagon | S1P₂ | EP₁ | EP₃ |
|---|---|---|---|---|---|
| | Receptor | Receptor | Receptor | Receptor | Receptor |
| | EC50 for | EC50 for | EC50 | EC50 for | EC50 for |
| | GLP-1 | Glucagon | for S1P | PGE₂ | PGE₂ |
| | (nM) | (nM) | (nM) | (nM) | (nM) |
| Luminescent | 2 | 0.1 | 7 | 10 | 4 |
| assay, Thaw/ | | | | | |
| ON | | | | | |
| Recovery | | | | | |
| Luminescent | 3 | 0.2 | 7 | 6 | 2 |
| Assay, | | | | | |
| Thaw/ | | | | | |
| Assay | | | | | |
| Fluorescent | 8 | 14 | 40 | 3 | 1 |
| Ca²⁺ assay | | | | | |

### Example 7

### Comparison of Luminescence Exhibited by Clytin K168D Induced by the Following GPCRs: CXCR1 Receptor, CXCR4 Receptor, GIP Receptor, GLP-1 Receptor, Glucagon Receptor, or EP1 Receptor

In another experiment, HEK293T cells were transiently co-transfected with plasmids containing a cDNA encoding an exemplary GPCR selected from one of: a GIP Receptor (Genbank Accession No. NM000164; obtained from OPEN BIOSYSTEMS); a CXCR1 Receptor (Genbank Accession No. M68932; obtained from OPEN BIOSYSTEMS); a CXCR4 Receptor (GenBank Accession No. M99293; obtained from OPEN BIOSYSTEMS); a Glucagon Receptor (see Example, 6); a GLP-1 Receptor (see Example 6); and an EP1 Receptor (see Example 6); a promiscuous G protein (e.g., in case of GIP Receptor, CXCR1 Receptor and CXCR4 Receptor) and a modified version of cytosolic Clytin having an amino acid substitution at position 168 of SEQ ID NO:1 (SEQ ID NO:9). At about 48 hours following transfection, the cells were cryopreserved with a CryoMed controlled rate freezer (THERMO SCIENTIFIC) and stored in liquid nitrogen.

In another experiment, CHO—K1 cells expressing the D2 Receptor and a promiscuous G protein (MILLIPORE, catalog no. HTS039C) were transfected with a modified version of cytosolic Clytin having an amino acid substitution at position 168 of SEQ ID NO:1 (SEQ ID NO:11). Cells containing stably integrated Clytin plasmid were selected by resistance to puromycin and subjected to limited dilution cloning to obtain clonal cell lines, from which a clone having highest dopamine-induced luminescence was chosen. Cells were cryopreserved with a CryoMed controlled rate freezer (THERMO SCIENTIFIC) and stored in liquid nitrogen.

The transfected and frozen cells were thawed and plated at 100,000 cells/well in a poly-D-lysine coated 96-well plate overnight in growth media consisting of DMEM/F12 containing 15% fetal bovine serum, non-essential amino acids, and penicillin/streptomycin. Cells were subsequently loaded with coelenterazine for 4 hours and assayed for GPCR-mediated bioluminescence, induced by a ligand for a Glucagon Receptor (Glucagon at 10⁻⁸ to 10^{-12.5} M), a GIP Receptor (GIP at 10⁻⁶ to 10⁻¹²), a GLP-1 Receptor (GLP-1 at 10⁻⁶ to 10⁻¹²), a CXCR1 Receptor (Interleukin-8 at 10⁻⁷ to 10⁻¹³ M), an EP₁ Receptor (prostaglandin E₂ at 10⁻⁶ to 10⁻¹¹ M), a CXCR4 Receptor (SDF-1α at 10⁻⁶ to 10⁻¹¹ M) or a D2 Receptor (dopamine at 10⁻⁴ to 10^{-10.5} M). The bioluminescence data depicted in the graphs was obtained on a FLIPRTetra Plus high throughput luminescent plate reader (MOLECULAR DEVICES).

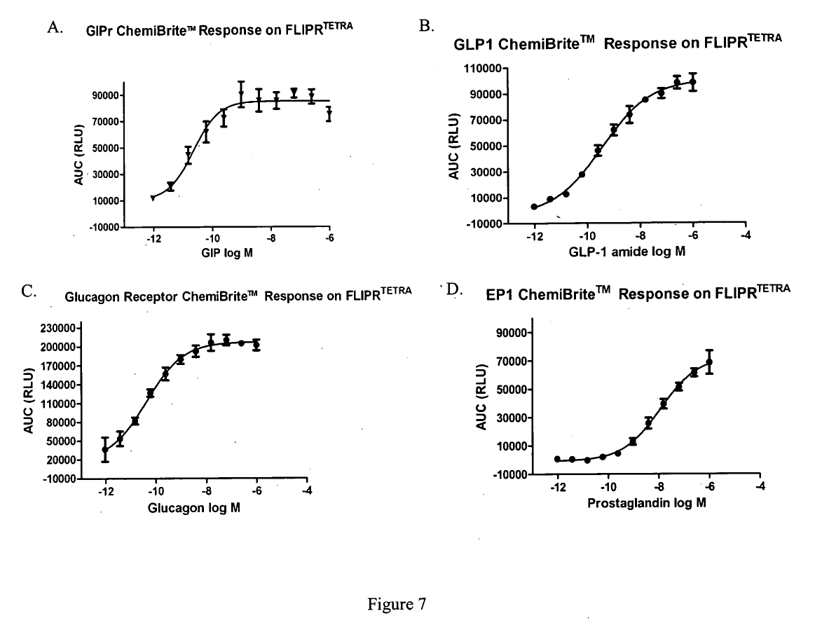

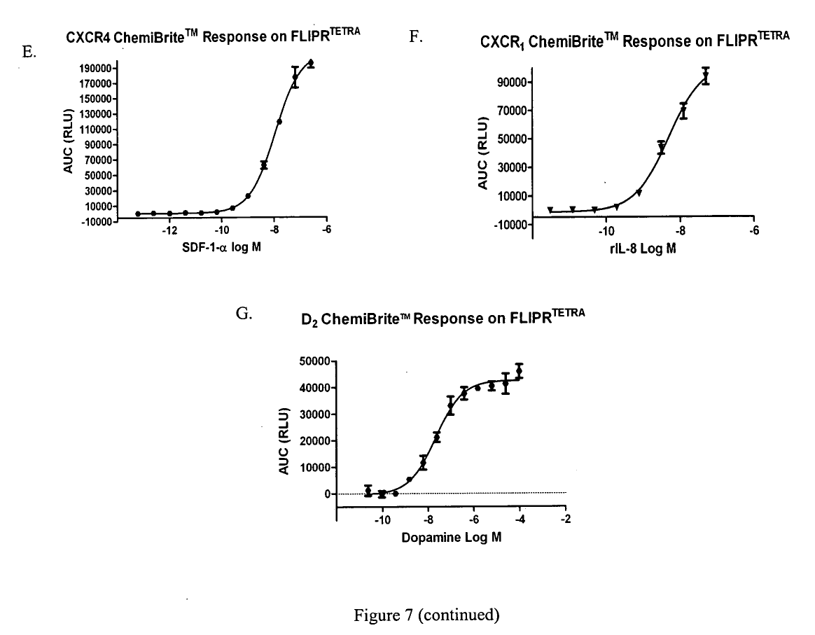

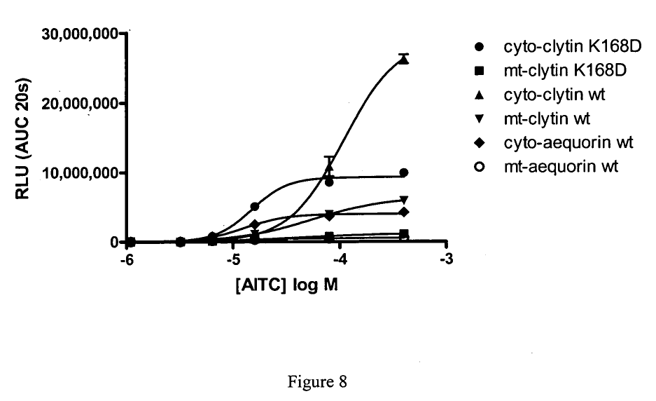

The results are depicted in FIGS. 7A-7G. The values obtained for the Glucagon receptor, the GLP-1 receptor and the EP₁ prostanoid receptor with the FLIPRTetra Plus plate reader are comparable to those obtained in FIG. 6 with a Victor2 plate reader (WALLAC, PERKINELMER).

### Example 8

### Comparison of Luminescence Exhibited by Various Photoprotein Variants, Induced by an Ion Channel

In another experiment, HEK293 cells stably expressing the TRPA1 cation channel (Accession No. NM_{—}007332; obtained from MILLIPORE) were transiently co-transfected with plasmids containing cDNAs encoding a panel of photoproteins, including mitochondrially targeted wild-type Clytin (mt-Clytin), modified mitochondrially targeted Clytin having a lysine to aspartic acid mutation at position 168 of SEQ ID NO:1 (SEQ ID NO:10), cytosolic wild-type Clytin (SEQ ID NO:1), modified cytosolic Clytin having a lysine to aspartic acid mutation at position 168 (SEQ ID NO:9), and mitochondrially targeted and cytosolic wild-type Aequorin (SEQ ID NOs:6 and 2, respectively).

The cells were aliquoted at 50,000 cells/well in a 96-well plate, and subsequently assayed for ion channel-mediated bioluminescence, induced by AITC ligand (10⁻⁶ to 10^{-3.5} M). The results of the dose response curve to varying concentrations of AITC is summarized in FIG. 7. Cytosolic Clytin K168D displays a lower EC50 than cytosolic wild-type Clytin. In addition, cytosolic Clytin K168D exhibits a higher signal than cytosolic and mitochondrial Aequorin. These data demonstrate that Clytin K168D allows enhanced sensitivity in analysis of an exemplary calcium-conducting ion channel.

The specification is most thoroughly understood in light of the teachings of the references cited within the specification which are hereby incorporated by reference. The embodiments within the specification provide an illustration of embodiments in this invention and should not be construed to limit its scope. The skilled artisan readily recognizes that many other embodiments are encompassed by this invention. All publications and inventions are incorporated by reference in their entirety. To the extent that the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with a true scope and spirit of the invention being indicated by the following claims.

What is claimed is:

1. A modified photoprotein comprising an amino acid sequence which comprises at least the lysine at position 168 of the amino acid sequence set forth in SEQ ID NO:1 replaced with an amino acid other than a histidine, arginine and lysine; wherein the modified photoprotein exhibits an increased affinity for intracellular calcium and enhanced bioluminescence relative to the photoprotein comprising the amino acid sequence set forth in SEQ ID NO:1.

2. A modified photoprotein comprising an amino acid sequence which comprises at least the lysine at position 168 of the amino acid sequence set forth in SEQ ID NO:1 replaced with an aspartic acid; wherein the modified photoprotein exhibits an increased affinity for intracellular calcium and enhanced bioluminescence relative to both a photoprotein comprising the amino acid sequence set forth in SEQ ID NO:1 and a photoprotein comprising the amino acid sequence set forth in SEQ ID NO:2.

3. The modified photoprotein of claim 1, wherein the modified photoprotein comprises an EC50 value of 500 nM or lower for intracellular calcium and wherein the modified photoprotein does not comprise the amino acid sequence set forth in SEQ ID NO:2 or a variant or derivative thereof.

4. The modified photoprotein of claim 1, comprising an amino acid sequence selected from the group consisting of SEQ ID NO:9 (K168D); SEQ ID NO:11 (K168E), SEQ ID NO:15 (K168G); SEQ ID NO:17 (K168N); SEQ ID NO:19 (K168Q); SEQ ID NO:21 (K168S); SEQ ID NO:23 (K168T); SEQ ID NO:25 (K168V) and SEQ ID NO:27 (K168Y).

5. The modified photoprotein of claim 1, wherein the amino acid modification is in the EF hand III domain of the photoprotein.

6. The modified photoprotein of claim 2, wherein the amino acid modification is in the EF hand III domain of the photoprotein.

7. The modified photoprotein of claim 1, wherein the modified photoprotein exhibits a 2%, or a 3%, or a 4%, or a 5%, or a 10%, or a 20%, or a 25%, or a 30%, or a 40%, or a 50%, or a 60%, or a 70%, or a 80%, or a 90%, or greater than 90% increase, in affinity for intracellular calcium relative to a photoprotein comprising the amino acid sequence set forth in SEQ ID NO:1.

8. The modified photoprotein of claim 2, wherein the modified photoprotein exhibits a 2%, or a 3%, or a 4%, or a 5%, or a 10%, or a 20%, or a 25%, or a 30%, or a 40%, or a 50%, or a 60%, or a 70%, or a 80%, or a 90%, or greater than 90% increase, in affinity for intracellular calcium relative to a photoprotein comprising the amino acid sequence set forth in SEQ ID NO:1.

9. The modified photoprotein of claim 1, wherein the modified photoprotein exhibits a 2%, or a 3%, or a 4%, or a 5%, or a 10%, or a 20%, or a 25%, or a 30%, or a 40%, or a 50%, or a 60%, or a 70%, or a 80%, or a 90%, or greater than 90% increase, in bioluminescence relative to a photoprotein comprising the amino acid sequence set forth in SEQ ID NO:1.

10. The modified photoprotein of claim 2; wherein the modified photoprotein exhibits a 2%, or a 3%, or a 4%, or a 5%, or a 10%, or a 20%, or a 25%, or a 30%, or a 40%, or a 50%, or a 60%, or a 70%, or a 80%, or a 90%, or greater than 90% increase, in bioluminescence relative to a photoprotein comprising the amino acid sequence set forth in SEQ ID NO:1.

11. The modified photoprotein of claim 1, wherein the affinity for intracellular calcium and bioluminescence are measured in a cell transfected with a nucleic acid molecule encoding the modified photoprotein of claim 1.

12. The modified photoprotein of claim 2, wherein the affinity for intracellular calcium and bioluminescence are measured in a cell transfected with a nucleic acid molecule encoding the modified photoprotein of claim 1.

13. The modified photoprotein of claim 1, wherein the affinity for intracellular calcium and bioluminescence are measured in a cell transfected with a nucleic acid molecule encoding the modified photoprotein of claim 2.

14. The modified photoprotein of claim 2, wherein the affinity for intracellular calcium and bioluminescence are measured in a cell transfected with a nucleic acid molecule encoding the modified photoprotein of claim 2.

15. The modified photoprotein of claim 11, wherein the cell is selected from a CHO cell, a HEK293T cell, a HeLa cell, an NIH3T3 cell and a U-2OS cell.

16. The modified photoprotein of claim 12, wherein the cell is selected from a CHO cell, a HEK293T cell, a HeLa cell, an NIH3T3 cell and a U-2OS cell.

17. The modified photoprotein of claim 13, wherein the cell is selected from a CHO cell, a HEK293T cell, a HeLa cell, an NIH3T3 cell and a U-2OS cell.

18. The modified photoprotein of claim 14, wherein the cell is selected from a CHO cell, a HEK293T cell, a HeLa cell, an NIH3T3 cell and a U-2OS cell.

19. A nucleic acid molecule encoding a modified photoprotein of claim 1.

20. A nucleic acid molecule encoding a modified photoprotein of claim 2.

21. A vector comprising the nucleic acid molecule of claim 19.

22. A vector comprising the nucleic acid molecule of claim 20.

23. A mammalian cell transfected with the vector of claim 21.

24. A mammalian cell transfected with the vector of claim 22.

25. An in vitro method for detecting calcium flux in a cell, the method comprising: a) providing a cell expressing a modified photoprotein of claim 1; b) contacting the cell with an agent which causes calcium flux; and c) detecting the photoprotein bioluminescence, wherein the bioluminescence is indicative of calcium flux.

26. An in vitro method for detecting calcium flux in a cell, the method comprising: a) providing a cell expressing a modified photoprotein of claim 2; b) contacting the cell with an agent which causes calcium flux; and c) detecting the photoprotein bioluminescence, wherein the bioluminescence is indicative of calcium flux.

27. A method for screening for compounds which modulate GPCR or ion channel activity, the method comprising: a) providing a cell expressing a modified photoprotein of claim 1; b) contacting the cell with a candidate compound; and c) detecting the photoprotein bioluminescence, wherein a change in the photoprotein bioluminescence in the presence of the candidate compound indicates that the compound modulates GPCR or ion channel activity.

28. A method for screening for compounds which modulate GPCR or ion channel activity, the method comprising: a) providing a cell expressing a modified photoprotein of claim 2; b) contacting the cell with a candidate compound; and c) detecting the photoprotein bioluminescence, wherein a change in the photoprotein bioluminescence in the presence of the candidate compound indicates that the compound modulates GPCR or ion channel activity.

29. The method of claim 25, wherein the calcium flux is caused by modulation of activity of any GPCR or ion channel.

30. The method of claim 26, wherein the calcium flux is caused by modulation of activity of any GPCR or ion channel.

31. The method of claim 27, wherein the GPCR is selected from the group consisting of an H₁ histamine receptor, a GIP receptor, a GLP-1 receptor, a glucagon receptor, an S1P₂ sphingosine 1-phosphate receptor, a CXCR1 chemokine receptor, a CXCR4 chemokine receptor, a D2 dopamine receptor, an EP₁ receptor, an EP₃ prostaglandin receptor and a TRPA1 cation channel.

32. The modified photoprotein of claim 1, comprising a mitochondrial targeting sequence at the N-terminus of the photoprotein.

33. The modified photoprotein of claim 2, comprising a mitochondrial targeting sequence at the N-terminus of the photoprotein.

34. The modified photoprotein of claim 32, wherein the mitochondrial targeting sequence comprises the amino acid sequence set forth in SEQ ID NO:8.

35. The modified photoprotein of claim 33, wherein the mitochondrial targeting sequence comprises the amino acid sequence set forth in SEQ ID NO:8.
